(19) United States
(12) Patent Application Publication
(10) Pub. No.: US 2024/0300891 A1
(43) Pub. Date: Sep. 12, 2024

(54) IONIZABLE LIPIDS, LIPID NANOPARTICLES FOR MRNA DELIVERY AND METHODS OF MAKING THE SAME

(71) Applicant: University of Cincinnati, Cincinnati, OH (US)

(72) Inventors: Joo-Youp Lee, Cincinnati, OH (US); Vishnu Sriram, Cincinnati, OH (US)

(73) Assignee: University of Cincinnati, Cincinnati, OH (US)

(21) Appl. No.: 18/578,361

(22) PCT Filed: Sep. 7, 2022
(86) PCT No.: PCT/US2022/042761

§ 371 Date: Jan. 11, 2024

Related U.S. Application Data
(60) Provisional application No. 63/220,817, filed on Jul. 12, 2021.
(60) Provisional application No. 63/390,747, filed on Jul. 20, 2022.

(51) Int. Cl. C07D 211/62; A61K 47/54; A61K 47/69

(52) U.S. Cl. CPC C07D 211/62; A61K 47/543; A61K 47/6929

(57) ABSTRACT

A composition including ionizable lipids is provided. Also provided is a composition forming lipid nanoparticle, wherein the composition includes the ionizable lipid, a helper lipid, a sterol, and a PEGylated lipid conjugate. Also provided are methods of making the ionizable lipids. The ionizable lipids can include 2AEOAP2, 2AEOAP4, 2AELAP2, 2AELAP4, Lipid 16A, Lipid 16B, Lipid 16C, Lipid 16B, and Lipid 20B.

### CROSS-REFERENCE TO RELATED APPLICATIONS

This application claims priority to U.S. Provisional Application Ser. No. 63/220,817, filed Jul. 12, 2021, and U.S. Provisional Application Ser. No. 63/390,747, filed Jul. 20, 2022, the contents of which are hereby incorporated by reference in their entirety.

### TECHNICAL FIELD

The present disclosure generally relates to ionizable lipids, lipid nanoparticles, and methods of making and using the same.

### BACKGROUND

One of the major challenges in the field of targeted delivery of biologically active substances is their instability and low cell penetrating potential, as well as their susceptibility to enzymatic degradation. This has created challenges in the development of therapies utilizing nucleic acid molecules, in particular RNA molecules.

In that respect, lipid-based nanoparticle compositions such as lipoplexes and liposomes have been used as packaging vehicles for biologically active substances to allow transport into cells and/or intracellular compartments. These lipid-based nanoparticle compositions typically comprise a mixture of different lipids such as ionizable lipids, helper lipids, structural lipids (such as sterols or cholesterol), and lipid conjugates.

Emerging clinical therapies, particularly nucleic acid-based vaccines, require drug delivery systems, such lipid nanoparticles, that can encapsulate and deliver a variety of cargo molecules. Accordingly, a need exists to develop new lipids and/or nanoparticles to better deliver the therapy.

### SUMMARY

In one embodiment, a composition comprising at least one ionizable lipid according to Formula (I) or a pharmaceutically-acceptable salt thereof, in which a) R1 is independently selected from

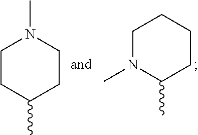

and R2 is selected from

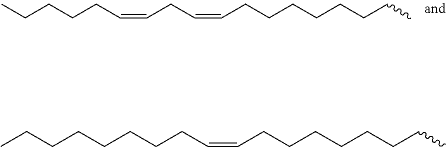

is provided. In embodiments, the ionizable lipid can be selected from the group consisting of: 2AEOAP2 , 2AEOAP4, 2AELAP2, and 2AELAP4.

In other embodiments, a composition comprising at least one ionizable lipid according to Formula (II), or a pharmaceutically-acceptable salt thereof, in which a) R1 is independently selected from

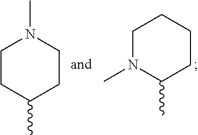

and b) R2 is selected from

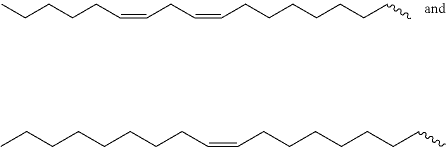

is provided. In embodiments, the ionizable lipid can be selected from the group consisting of Lipid 16A, Lipid 16B, Lipid 16C, and Lipid 16D.

In other embodiments, a composition comprising Lipid 20B is provided.

In embodiments, the composition of any of the previous compositions may further include a helper lipid; a sterol; and a PEGylated lipid conjugate, wherein the composition forms lipid nanoparticles. In embodiments the ionizable lipid is selected from the group consisting of: 2AEOAP2, 2AEOAP4, 2AELAP2, 2AELAP4, Lipid 16A, Lipid 16B, Lipid 16C, Lipid 16B, and Lipid 20B. In embodiments, the helper lipid can be selected from the group consisting of: 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dioleyl-sn-glycero-3-phosphotidylcholine (DOPC) 1,2-dipalmitoyl-sn-glycero-3-phosphoethanolamine (DPPE), 1,2-dimyristoyl-sn-glycero-3-phosphoethanolamine (DMPE), and 1,2-dioleoyl-sn-glycero-3-phospho-(1′-rac-glycerol) (DOPG). In embodiments, the helper lipid is DOPE. In embodiments, the sterol is cholesterol or a derivative thereof. In embodiments, the PEGylated lipid conjugate is PEGylated myristoyl diglyceride (PEG-DMG). In embodiments, the lipid nanoparticle at least partially encapsulates a nucleic acid. In embodiments, wherein the nucleic acid is mRNA. In embodiments, the composition can further include a pharmaceutically acceptable excipient. In embodiments, the composition is formulated for administration by injection or infusion. In embodiments, the ionizable lipid can include from about 40-60% mole percent, the helper lipid comprises from about 10-20% molar percent, the sterol comprises from about 30-50% mole percent; and the conjugate lipid comprises from about 1-5% mole percent.

These and other features, aspects, and advantages of the present invention will become better understood with reference to the following description, appended claims, and accompanying drawings.

### BRIEF DESCRIPTION OF THE DRAWINGS

Though the specification concludes with claims particularly pointing out and distinctly claiming the invention, it is believed that the present invention will be better understood from the following description taken in conjunction with the accompanying drawings, in which:

FIG. 1 depicts an illustrative embodiment of the synthesis of an ionizable imidazole lipid, according to one or more aspects shown and described herein;

FIG. 2 depicts an ¹H NMR spectrum of a first intermediate product (Product 1) in the synthesis of the ionizable lipid of FIG. 1, according to one or more aspects shown and described herein;

FIG. 3 depicts an ¹H NMR spectrum of a second intermediate product (Product 2) in the synthesis of the ionizable lipid of FIG. 1, according to one or more aspects shown and described herein;

FIG. 4 depicts an ESI-MS spectrum of the ionizable lipid of FIG. 1, having a theoretical mass of 782.59 and an observed mass of 782.59, according to one or more aspects shown and described herein;

FIG. 5A depicts a graphical representation of the particle size of various lipid nanoparticles according to one or more aspects shown and described herein;

FIG. 5B depicts a graphical representation of the surface charge of various lipid nanoparticles according to one or more aspects shown and described herein;

FIG. 6 depicts a graphical representation of the in vitro cell uptake and transfection efficiency of various lipid nanoparticles in Hela cells according to one or more aspects shown and described herein;

FIG. 7 depicts a graphical representation of the in vitro cell uptake and transfection efficiency of various lipid nanoparticles in Jurkat cells according to one or more aspects shown and described herein;

FIG. 8 depicts an illustrative embodiment of the synthesis of Product 4, according to one or more aspects shown and described herein;

FIG. 9 depicts an ¹H NMR spectrum of an intermediate product (Product 3) in the synthesis of Product 4 of FIG. 8, according to one or more aspects shown and described herein;

FIG. 10 depicts an ¹H NMR spectrum of Product 4, according to one or more aspects shown and described herein;

FIG. 11 depicts an illustrative embodiment of the synthesis of Product 5, an ionizable lipid 2AEOAP2, according to one or more aspects shown and described herein;

FIG. 12 depicts an illustrative embodiment of the synthesis of Product 6, according to one or more aspects shown and described herein;

FIG. 13 depicts an illustrative embodiment of the synthesis of Product 7, according to one or more aspects shown and described herein;

FIG. 14 depicts an illustrative embodiment of the synthesis of Product 8, according to one or more aspects shown and described herein;

FIG. 15 depicts an illustrative embodiment of the synthesis of a lipid with a linoleyl alcohol tail, according to one or more aspects shown and described herein;.

FIG. 16 depicts an ¹H NMR spectrum of an intermediate product (Product 9) in the synthesis of the lipid with the linoleyl alcohol tail of FIG. 15, according to one or more aspects shown and described herein;

FIG. 17 depicts an ¹H NMR spectrum of the lipid with the linoleyl alcohol tail of FIG. 15, according to one or more aspects shown and described herein;

FIG. 18 depicts an illustrative embodiment of the synthesis of a lipid (Product 11) according to one or more aspects shown and described herein;

FIG. 19 depicts an illustrative embodiment of the synthesis of a lipid (Product 12) according to one or more aspects shown and described herein;

FIG. 20 depicts an illustrative embodiment of the synthesis of a lipid (Product 13) according to one or more aspects shown and described herein;

FIG. 21 depicts an illustrative embodiment of the synthesis of a lipid (Product 14) according to one or more aspects shown and described herein;

FIG. 22A depicts a graphical representation of the particle size of various lipid nanoparticles according to one or more aspects shown and described herein;

FIG. 22B depicts a graphical representation of the surface charge surface charge of various lipid nanoparticles according to one or more aspects shown and described herein;

FIG. 23 depicts a graphical representation of cell uptake and transfection efficiency of various lipid nanoparticle formulations in Jurkat cells, according to one or more aspects shown and described herein;

FIG. 24A depicts an illustrative embodiment of the synthesis of ring lipids, according to one or more aspects shown and described herein;

FIG. 24B depicts illustrative examples of four ring lipids designated Lipids 16A-16D, synthesized with the scheme of FIG. 24A according to one or more aspects shown and described herein;

FIG. 25A depicts an illustrative embodiment of the synthesis of piperidine-based lipids according to one or more aspects shown and described herein; and

FIG. 25B depicts illustrative examples of piperidine-based lipids designated Lipids 20A-20B, synthesized with the scheme of FIG. 25A, according to one or more aspects shown and described herein.

The exemplifications set out herein illustrate at least one embodiment of the present disclosure, and such exemplifications are not to be construed as limiting the scope of the present disclosure in any manner.

### DETAILED DESCRIPTION

Features and advantages of the invention will now be described with occasional reference to specific embodiments. However, the invention may be embodied in different forms and should not be construed as limited to the embodiments set forth herein. Rather, these embodiments are provided so that this disclosure will be thorough and complete and will fully convey the scope of the invention to those skilled in the art.

Unless otherwise defined, all technical and scientific terms used herein have the same meaning as commonly understood by one of ordinary skill in the art to which the invention belongs. The terminology used in the description herein is for describing particular embodiments only and is not intended to be limiting.

As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. It is to be further understood that where descriptions of various embodiments use the term “comprising,” and/or “including” those skilled in the art would understand that in some specific instances, an embodiment can be alternatively described using language “consisting essentially of” or “consisting of.” The term “or a combination thereof” means a combination including at least one of the foregoing elements.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. One of ordinary skill in the art will understand that any numerical values inherently contain certain errors attributable to the measurement techniques used to ascertain the values.

It should be understood that every numerical range given throughout this specification will include every narrower numerical range that falls within such broader numerical range, as if such narrower numerical ranges were all expressly written herein. Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

As used herein, the terms “improve,” “increase,” “inhibit,” “reduce,” or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

The term “independently selected from,” as used herein, is intended to mean that the referenced groups can be the same, different, or a mixture thereof, unless the context clearly indicates otherwise. Thus, under this definition, the phrase “X¹, X², and X³ are independently selected from noble gases” would include the scenario where X¹, X², and X³ are all the same, where X¹, X², and X³ are all different, and where X¹ and X² are the same but X³ is different.

The term “subject” as used herein refers to any living organism to which a pharmaceutical can be administered. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult, child, and newborn subjects, as well as fetuses, whether male or female, are intended to be covered.

As used herein, the term “pharmaceutically acceptable” refers to approved or approvable by a regulatory agency of the Federal or a state government or listed in the U.S. Pharmacopeia or other generally recognized pharmacopeia for use in animals, including humans.

As used herein, the term “pharmaceutically acceptable excipient, carrier, or diluent” or the like refer to an excipient, carrier, or diluent that can be administered to a subject, together with an agent, and which does not destroy the pharmacological activity thereof and is nontoxic when administered in doses sufficient to deliver a therapeutic amount of the agent.

The term “pharmaceutically acceptable salt” as used herein refers to pharmaceutically acceptable organic or inorganic salts of an ionizable lipid of the present disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g, sodium and potassium) salts, alkaline earth metal (e.g, magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

As used herein, the term “lipid encapsulated” is meant to refer to a lipid particle that provides an active agent or therapeutic agent, such as a nucleic acid (e.g, an anti-sense oligonucleotide (ASO), mRNA, siRNA, close ended DNA (ceDNA), viral vector, etc.), with full encapsulation, partial encapsulation, or both. In a preferred embodiment, the nucleic acid is fully encapsulated in the lipid particle (e.g., to form a nucleic acid containing lipid particle).

Unless otherwise stated, the structures depicted and described herein include all isomeric (e.g., enantiomeric, diastereomeric, and geometric) forms of the structure; for example, tautomers, R and S configurations for each asymmetric center, Z and E double bond isomers, and Z and E conformational isomers. Additionally, unless otherwise stated, the structures depicted and described herein include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools or as therapeutic agents.

### Ionizable Lipids

As used herein, the term “ionizable lipid” is refers to a lipid, e.g., cationic lipid, having at least one protonatable or deprotonatable group, such that the lipid is positively charged at a pH at or below physiological pH (e.g., pH 7.4), and neutral at a second pH, at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. In some aspects, an ionizable lipid is characterized by three portions: an amine head, a linker and a hydrophobic tail.

In some aspects, ionizable lipids of the present disclosure are synthesized by reacting an acyl chloride or mesyl chloride with trimethylamine in dichloromethane and a fatty alcohol to generate a first reaction product. The reaction can be performed with or without stirring. The reaction may proceed for any appropriate amount of time with or without monitoring. In aspects, the amount of time includes, for example, about 0-96 hours, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, and 96, or any range having endpoints defined by any two of the aforementioned values.

Illustrative acyl chlorides include, but are not limited to, acrylol chloride, acetyl chloride, adipoyl chloride, anisoyl chloride, azelaoyl chloride, benzoyl chloride, butyryl chloride, chloroacetyl chloride, glutaryl chloride, hepatnoyl chloride, hexanoyl chloride, isobutryrl chlorid, layroyl chloride, malonyl chloride, methacrylol chloride, octanoyl chloride, oxalyl chloride, pentanoyl chloride, pimeloyl chloride, pivaloyl chloride, propionyl chloride, thionyl chloride, thioacyl chloride, and the like, though any suitable acyl chloride is contemplated and possible.

Illustrative fatty alcohols include, but are not limited to, oleyl alcohol, linoleyl alcohol, tert-butyl alcohol, tert-amyl alcohol, enanthic alcohol, capryl alcohol, pelargoinc alcohol, capric alcohol, undecyl alcohol, lauryl alcohol, tridecyl alcohol, myristyl alcohol, pentadecyl alcohol, cetyl alcohol, palmitoleyl alcohol, heptadecyl alcohol, stearyl alcohol, nonadecyl alcohol, arachidyl alcohol, heneicosyl alcohol, behenyl alcohol, erucyl alcohol, lignoceryl alcohol, and ceryl alcohol, though any suitable fatty alcohol is contemplated and possible.

In aspects the first reaction product can be extracted using an appropriate solvent. Illustrative examples of solvents include, but are not limited to, polar aprotic solvents (e.g. dichloromethane (DCM), dimethyl sulfoxide (DMSO), ethyl acetate, acetone, dimethylformamide (DMF), acetonitrile, nitromethane, propylene carbonate, etc.), nonpolar hydrocarbon solvents (e.g. pentane, hexane, benzene, heptane, toluene, etc.), nonpolar ether solvents (e.g. diethyl ether, tetrahydrofuran, etc.), nonpolar chlorocarbon solvents (e.g. chloroform, etc.), polar protic solvents (e.g. ammonia, formic acid, n-butanol, isopropyl alcohol, n-propanol, ethanol, methanol, acetic acid, water etc.), or combinations thereof.

Monitoring of the reaction may include, for example, thin-layer chromatography, Fourier-transform infrared spectroscopy (FTIR), Ultraviolet-visible spectroscopy (UV-Vis), nuclear magnetic resonance (NMR), temperature monitoring, pH monitoring, and the like, though any method of monitoring known in the art is contemplated and possible.

In some aspects, the first reaction product is allowed to dry after extraction. In aspects, drying of the product is effected by any acceptable method, including, but not limited to, evaporation at ambient temperature, use of a heat source (e.g., a steam bath, hot plate, sand bath, oven, etc.), rotary evaporation, or gas blow-down.

In some embodiments, the first reaction product is (9Z)-9-octadecen-1-yl 2-propenoate or 2-propenoic acid, 9,12-octadecadienyl ester, (Z,Z)-(9CI).

In aspects, the first reaction product is reacted with an amino alcohol to generate a second reaction product. In aspects, this reaction is performed with stirring. In other aspects, it is performed without stirring. In aspects, this reaction can occur at temperatures above ambient temperature, including, but not limited to, about 30, 35, 40, 45, 50, 55, 60, 65, 70, 75, 80, 85, 90, 95, or 100° C. In aspects, this reaction can occur at about ambient temperature. In aspects, this reaction can occur below ambient temperature, including, but not limited to, about 20, 15, 10, 5, or 0° C. The reaction may proceed for any appropriate amount of time with or without monitoring. Illustrative amino alcohols include, but are not limited to ethanolamines, (i.e. 2-aminoethanol), methanolamine, dimethylethanolamine, N-methylethanolamine, and aminomethyl propanol.

One or more solvent are added to extract the second reaction product. In aspects, the second reaction product is allowed to dry. In some embodiments, the second reaction product is β-alanine, N-(2-hydroxyethyl)-N-[3-(9-octadecenyloxy)-3-oxopropyl]-9-octadecenyl ester (Z,Z)-(9CI) (Product 4) or Product 10, having the structure:

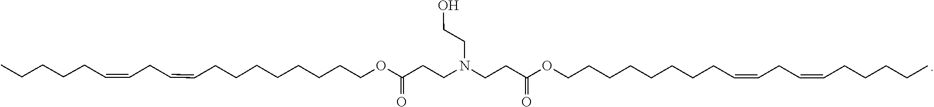 [SMILES: CCCCC/C=C\C/C=C\CCCCCCCCOC(=O)CCN(CCO)CCC(=O)OCCCCCCCC/C=C\C/C=C\CCCCC]

In aspects, the second reaction product is used in the synthesis of an ionizable lipid. The second reaction product can be dissolved in a solution of one or more solvents. In embodiments, the solvents are DCM and DMF. In aspects, a carboxyl activating agent, such as 1-ethyl-3-(3-dimethylaminopropyl)carbodiimide (EDC) or N,N′-dicyclohexyl carbodiimide (DCC), and an esterification catalyst, such as 4-dimethylaminopyridine (DMAP) are added to the solution. The reaction can be performed with or without stirring. The reaction may proceed for any appropriate amount of time with or without monitoring.

In aspects, a carboxylic acid derivative is added following the addition of the carboxyl activating agent and esterification catalyst. Illustrative carboxylic acid derivatives include, but are not limited to, 1-methylpiperidine-2-carboxylic acid hydrochloride, 1-methyl-4-piperidinecarboxylic acid, 3-(dimethylamino) propionic acid hydrochloride, 4-dimethylaminobutyric acid hydrochloride, or 1H-imidazol-1-ylacetic acid. The carboxylic acid derivative reaction can be performed with or without stirring. The reaction may proceed for any appropriate amount of time with or without monitoring. The solvent is evaporated to yield the ionizable lipid.

In other aspects, a reaction product can be synthesized by mixing a carboxyl activating agent, such as 1-ethyl-3-(3-dimethylaminopropyl)carbodiimide (EDC) or N,N′-dicyclohexyl carbodiimide (DCC), and an esterification catalyst, such as 4-dimethylaminopyridine (DMAP) to a solution of one or more solvents and a cyclitol, such as cyclohexane-1,3,5-triol. In aspects, a carboxylic acid derivative is also added. The reaction can be performed with or without stirring. The reaction may proceed for any appropriate amount of time with or without monitoring. The desired reaction product(s) can be separated by any method known in the art, such as column chromatography, to separate the reaction product.

In embodiments, the reaction product is Product 15:

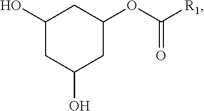

wherein R1 is

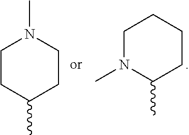

In aspects, the reaction product is used to synthesize an ionizable lipid. In aspects, the reaction product is mixed with a carboxyl activating agent and an esterification catalyst. A fatty alcohol can may be added to the solution. The reaction can be performed with or without stirring. The reaction may proceed for any appropriate amount of time with or without monitoring. The reaction mixture may be extracted using a solvent. The desired ionizable lipid can be separated from the extracted mixture using any appropriate separation method, such as column chromatography and the like.

In other aspects, a first reaction product is synthesized by reacting a fatty alcohol with mesyl chloride (e.g., methanesulfonyl chloride) in the presence of triethlamine in dichloromethane. The first reaction product can be extracted using a solvent.

In embodiments, the first reaction product is Product 17:

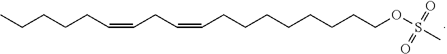 [SMILES: CCCCC/C=C\C/C=C\CCCCCCCCOS(C)(=O)=O]

The first reaction product may be dissolved in a solvent, such as diethyl ether, and magnesium bromide ethyl etherate may be added to the solution. The second reaction product can be extracted using one or more solvents.

In embodiments, the second reaction product is product 18:

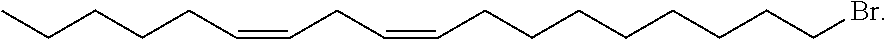 [SMILES: CCCCC/C=C\C/C=C\CCCCCCCCBr]

The second reaction product can be added dropwise to a mixture of magnesium turnings in a solvent, causing an exothermic reaction. In aspects, iodine may be added to initiate the reaction. After completion, ethyl formate can be added dropwise to the solution. The reaction mixture can be extracted using any appropriate solvent and separated using column chromatography. The reaction system can be degassed using nitrogen and reflux condensation, to yield the third reaction product.

In embodiments, the third reaction product is product 19:

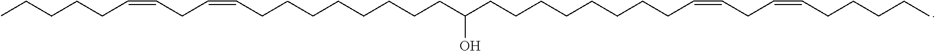 [SMILES: CCCCC/C=C\C/C=C\CCCCCCCCC(O)CCCCCCCC/C=C\C/C=C\CCCCC]

In aspects, the third reaction product may be reacted with a carboxyl activating agent, an esterification catalyst, and a carboxylic acid derivative to synthesize the ionizable lipid.

In embodiments, the ionizable lipids have a structure according to Formula (I):

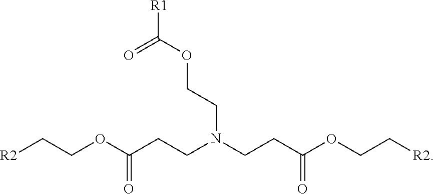

In embodiments, R1 is independently selected from

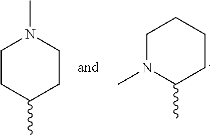

In embodiments, R2 is independently selected from

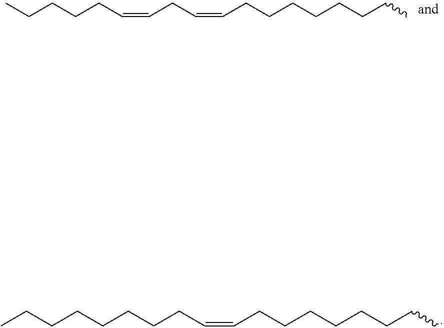

In embodiments, the ionizable lipids have a structure according to Formula (II):

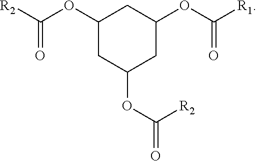

In embodiments, R1 is independently selected from

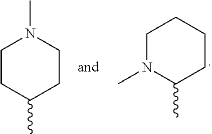

In embodiments, R2 is independently selected from

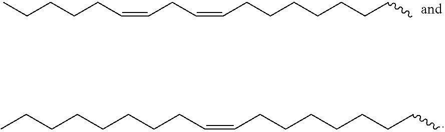

In embodiments, the ionizable lipids have a structure according to Formula (III):

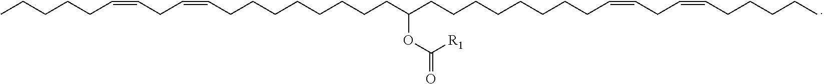

In embodiments, R1 is independently selected from

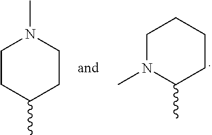

In embodiments, R1 is

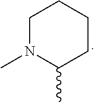

According to some embodiments of any of the aspects or embodiments herein, the ionizable lipid is selected from any one of the lipids in Table 1 or a pharmaceutically acceptable salt thereof.

TABLE 1

| Lipid Name | Structure |
|---|---|
| 2AEOAP2 | 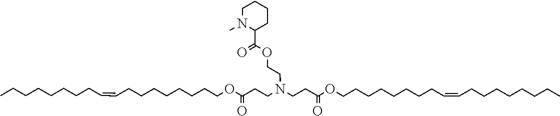 |
| 2AEOAP4 | 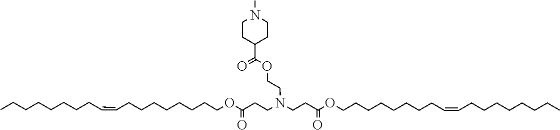 [SMILES: CCCCCCCC/C=C\CCCCCCCCOC(=O)CCN(CCOC(=O)C1CCN(C)CC1)CCC(=O)OCCCCCCCC/C=C\CCCCCCCC] |
| 2AEOAD2 | 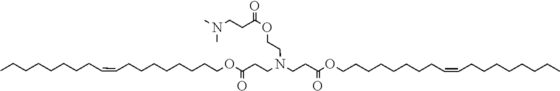 [SMILES: CCCCCCCC/C=C\CCCCCCCCOC(=O)CCN(CCOC(=O)CCN(C)C)CCC(=O)OCCCCCCCC/C=C\CCCCCCCC] |
| 2AEOAD3 | 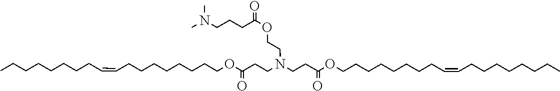 [SMILES: CCCCCCCC/C=C\CCCCCCCCOC(=O)CCN(CCOC(=O)CCCN(C)C)CCC(=O)OCCCCCCCC/C=C\CCCCCCCC] |
| 2AELAP2 | 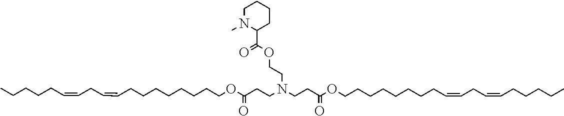 [SMILES: CCCCC/C=C\C/C=C\CCCCCCCCOC(=O)CCN(CCOC(=O)C1CCCCN1C)CCC(=O)OCCCCCCCC/C=C\C/C=C\CCCCC] |
| 2AELAP4 | 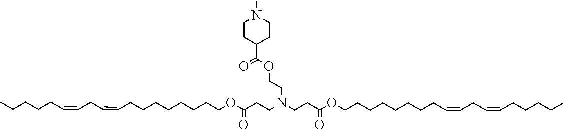 [SMILES: CCCCC/C=C\C/C=C\CCCCCCCCOC(=O)CCN(CCOC(=O)C1CCN(C)CC1)CCC(=O)OCCCCCCCC/C=C\C/C=C\CCCCC] |
| 2AELAD2 | 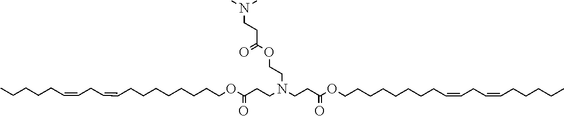 [SMILES: CCCCC/C=C\C/C=C\CCCCCCCCOC(=O)CCN(CCOC(=O)CCN(C)C)CCC(=O)OCCCCCCCC/C=C\C/C=C\CCCCC] |
| 2AELAD3 | 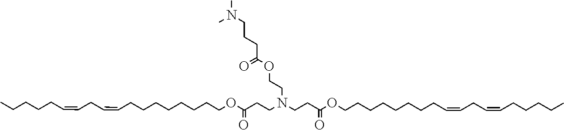 [SMILES: CCCCC/C=C\C/C=C\CCCCCCCCOC(=O)CCN(CCOC(=O)CCCN(C)C)CCC(=O)OCCCCCCCC/C=C\C/C=C\CCCCC] |
| Lipid 16A | 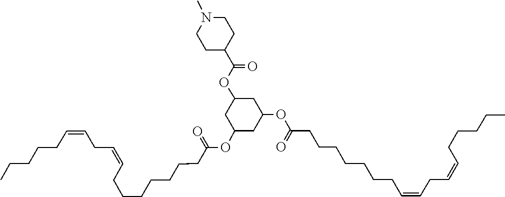 [SMILES: CCCCC/C=C\C/C=C\CCCCCCCCC(=O)OC1CC(OC(=O)CCCCCCC/C=C\C/C=C\CCCCC)CC(OC(=O)C2CCN(C)CC2)C1] |
| Lipid 16B | 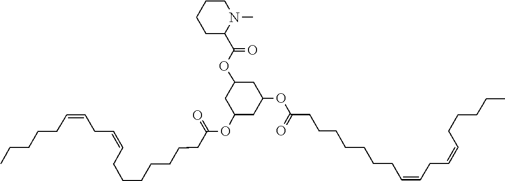 [SMILES: CCCCC/C=C\C/C=C\CCCCCCCCC(=O)OC1CC(OC(=O)CCCCCCC/C=C\C/C=C\CCCCC)CC(OC(=O)C2CCCCN2C)C1] |
| Lipid 16C | 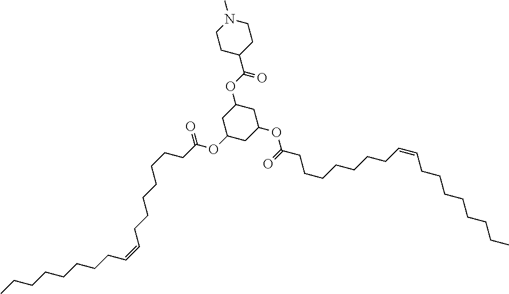 [SMILES: CCCCCCCC/C=C\CCCCCCCC(=O)OC1CC(OC(=O)CCCCCCC/C=C\CCCCCCCC)CC(OC(=O)C2CCN(C)CC2)C1] |
| Lipid 16D | 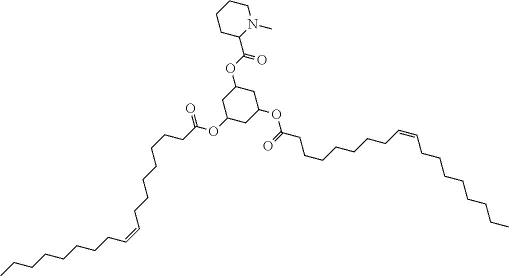 [SMILES: CCCCCCCC/C=C\CCCCCCCC(=O)OC1CC(OC(=O)CCCCCCC/C=C\CCCCCCCC)CC(OC(=O)C2CCCCN2C)C1] |
| Lipid 20A | 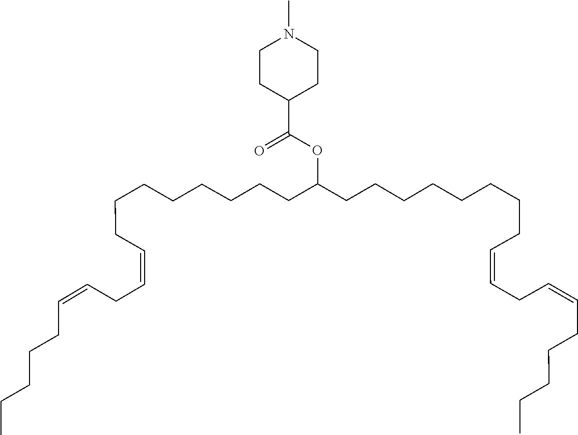 [SMILES: CCCCC/C=C\C/C=C\CCCCCCCCC(CCCCCCCC/C=C\C/C=C\CCCCC)OC(=O)C1CCN(C)CC1] |
| Lipid 20B | 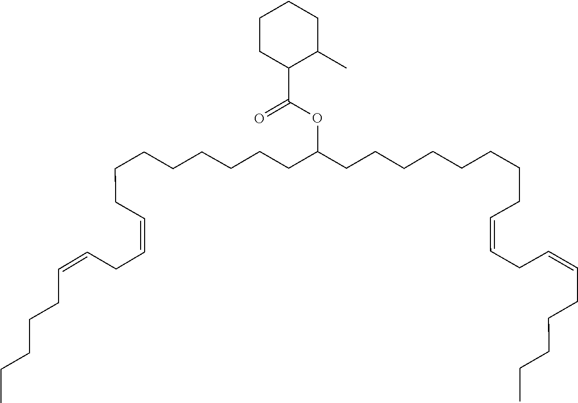 [SMILES: CCCCC/C=C\C/C=C\CCCCCCCCC(CCCCCCCC/C=C\C/C=C\CCCCC)OC(=O)C1CCCCC1C] |
| Lipid 20C | 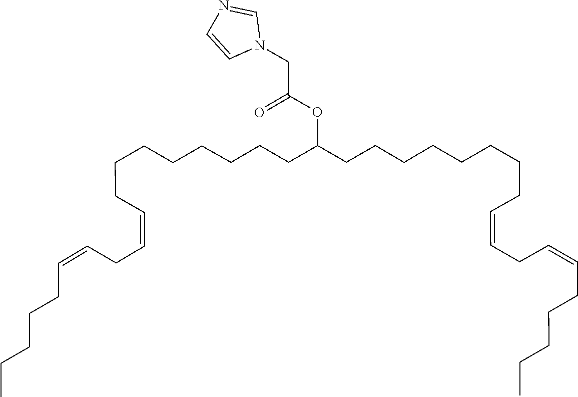 [SMILES: CCCCC/C=C\C/C=C\CCCCCCCCC(CCCCCCCC/C=C\C/C=C\CCCCC)OC(=O)Cn1ccnc1] |

### Lipid Nanoparticles

In some aspects of this disclosure, the ionizable lipids disclosed herein, particularly those identified in Table 1 may be incorporated into lipid nanoparticles (LNPs). In some aspects, the lipid nanoparticles may be used to deliver cargo molecules (e.g. polypeptides, nucleic acids, small molecules, etc.) alone or as packaged in a deliverable pharmaceutical composition, such as a vaccine. Lipid nanoparticles may include one or more ionizable lipids, helper lipids, sterol and/or conjugated lipid components along with nucleic acid or polypeptide cargo of interest.

In some aspects, lipid nanoparticles comprise one or more ionizable lipids as described herein. In some embodiments, lipid nanoparticles comprise one or more helper lipids as described herein. In some embodiments, lipid nanoparticles comprise one or more sterols as described herein. In some embodiments, lipid nanoparticles comprise one or more conjugate-linker lipids as described herein.

LNPs may be used in some aspects to carry and/or deliver cargo to a subject or a portion thereof such as a cell or cellular compartment. DNA and RNA vaccines utilizing such LNPs share many similarities, but each targets different cellular environments. For example, DNA vaccines target and are used in the nucleus of a cell, whereas RNA vaccines target and are expressed in the cytosol. This makes mRNA vaccines easier to deliver, yet both may capitalize on the success of recent advances in LNP formulations and sometimes other modifications to the nucleic acid cargo itself that may improve overall function.

As used herein, the term “nanoparticle” refers to a particle having dimensions on a scale of less than about 1000 nm. Routinely, nanoparticles have any one structural feature on a scale of less than about 500 nm, less than about 400 nm, less than about 300 nm, less than about 200 nm or less than about 100 nm. In exemplary embodiments, a nanoparticle is a particle having one or more dimensions of the order of about 10-500 nm. In other exemplary embodiments, a nanoparticle is a particle having one or more dimensions of the order of about 10-1000 nm. A spherical nanoparticle would have a diameter, for example, of between 10-100 nm or 10-1000 nm.

The term “particle size” or “particle diameter” refers to the mean diameter of the particles in a sample, as measured by dynamic light scattering (DLS), multiangle light scattering (MALS), nanoparticle tracking analysis, or comparable techniques. It will be understood that a dispersion of lipid nanoparticles as described herein will not be of uniform size but can be described by the average diameter and, optionally, the polydispersity index.

In some embodiments, lipid nanoparticles described herein can have an average particle diameter that is about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185, nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285, nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385, nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485, nm, 490 nm, 495 nm, 500 nm, or any range having endpoints defined by any two of the aforementioned values. For example, in some embodiments, lipid nanoparticles described herein have an average particle diameter from between 100 nm to 200 nm.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −20 mV to about +20 mV, from about −20 mV to about +15 mV, from about −20 mV to about +10 mV, from about −20 mV to about +5 mV, from about −20 mV to about 0 mV, from about −20 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, lipid nanoparticles contain a mixture of ionizable and/or cationic lipids in combination with any of the above ionizable lipids for the formation of lipid nanoparticles. Suitable cationic lipids include, but are not limited to N-[1-(2,3-dioleyloxy)propyl]-N,N,N-trimethylammonium chloride (DOTMA), 5-carboxyspermylglycinedioctadecylamide (DOGS) 2,3-dioleyloxy-N-[2(spermine-carboxamido)ethyl]-N,N-dimethyl-1-propanaminium (DOSPA), 1,2-Dioleoyl-3-Dimethylammonium-Propane (DODAP), 1,2-Dioleoyl-3-Trimethylammonium-Propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′, 1-2′-octadecadienoxy)propane (CpLinDMA), N,N-dimethyl-3,4-ioleyloxybenzylamine (DMOBA), 1,2-N,N′-dioleylcarbamyl-3-dimethylaminopropane or (DOcarbDAP_, 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-DMA), 2,2-dilinoleyl-4-dimethylaminoethyl-[1,3]-dioxolane (DLin-K-XTC2-DMA), 2-(2,2-di((9Z,12Z)-octadeca-9,1 2-dien-1-yl)-1,3-dioxolan-4-yl)-N,N-dimethylethanamine (DLin-KC2-DMA), DLin-MC3-DMA, (6Z,9Z,28Z,31Z)-heptatriaconta-6,9,28,31-tetraen-19-yl4-(dimethylamino) butanoate (MC3), or mixtures thereof.

In some embodiments, ionizable lipids constitute at least about 5%, 10%, 20%, 30%, 35%, 40%, 45%, 50%, 55%, 60%, 65%, 70%, or 75% of the total lipids in a suitable lipid solution by weight or by molar. In some embodiments, ionizable lipid(s) constitute(s) about 30-70% (e.g., about 30-65%, about 30-60%, about 30-55%, about 30-50%, about 30-45%, about 30-40%, about 35-50%, about 35-45%, about 35-40%, about 40-60%, about 45-60%, about 50-60%, about 55-60, about 40-65%, about 45-65%, about 50-65%, about 55-65%, about 60-65%) of the total lipid mixture by weight or by molar.

In embodiments, the LNPs can further comprise a non-cationic, helper lipid. The helper lipid can serve to increase fusogenicity and/or increase stability of the LNP during formation. Helper lipids include amphipathic lipids, neutral lipids and anionic lipids. As used herein, the phrase “helper lipid” refers to any neutral, zwitterionic or anionic lipid. Accordingly, the non-cationic lipid can be a neutral uncharged, zwitterionic, or anionic lipid. As used herein, the term “neutral lipid” is meant to refer to any of a number of lipid species that exist either in an uncharged or neutral zwitterionic form at a selected pH. At physiological pH, such lipids include, but are not limited to, for example, diacylphosphatidylcholine, diacylphosphatidylethanolamine, ceramide, sphingomyelin, cephalin, cholesterol, cerebrosides, and diacylglycerols.

As used herein, the term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Helper lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), 2-diphytanoyl-sn-glycero-3-phosphatidylethanolamine (DPyPE), distearoyl-sn-glycero-phosphoethanolamine, palmitoyloleoylphosphatidylcholine (POPC), palmitoyloleoyl-phosphatidylethanolamine (POPE), diolcoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), egg sphingomyelin (ESM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, or a combinations thereof.

In some embodiments, the one or more helper lipids are selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)).

In some embodiments, helper lipids may constitute at least about 5%, 10%, 15%, 20%, 25%, 30%, 35%, 40%, 45%, or 50% of the total lipids in a suitable lipid solution by weight or by molar. In some embodiments, helper lipid(s) constitute(s) about 5-25% (e.g., about 5-20%, about 5-15%, about 5-10%, about 10-25%, about 10-20%, about 10-15%, about 15-25%, about 15-20%, or about 20-25%) of the total lipids in a suitable lipid solution by weight or by molar.

In some embodiments, the LNPs can further comprise a component, such as a sterol, to provide membrane integrity and stability of the lipid particle. Illustrative examples of sterols include, but are not limited to, cholesterol, ergosterol, campesterol, oxysterol, antrosterol, desmosterol, nicasterol, sitosterol, stigmasterol, derivatives and variants thereof, and mixtures of the foregoing. In some embodiments, the sterol is cholesterol or a derivative or variant thereof. Non-limiting examples of cholesterol derivatives include 5a-cholestanol, 5P-coprostanol, cholesteryl-(2′-hydroxy)-ethyl ether, cholesteryl-(4′-hydroxy)-butyl ether, 6-ketocholestanol; 5a-cholestane, cholestenone, 5a-cholestanone, 5P-cholestanone, cholesteryl decanoate, 25-hydroxycholesterol (25-OH), 20a-hydroxycholesterol (20a-OH), 27-hydroxycholesterol, 6-keto-5a-hydroxycholesterol, 7-ketocholesterol, 7-hydroxycholesterol, 7a-hydroxycholesterol, 7-25-dihydroxycholesterol, beta-sitosterol, stigmasterol, brassicasterol, campesterol, or combinations thereof.

In some embodiments, sterols constitute at least about 5%, 10%, 20%, 30%, 40%, 50%, 60%, or 70% of the total lipids in a suitable lipid solution by weight or by molar. In some embodiments, sterols constitute about 30-50% (e.g., about 30-45%, about 30-40%, about 35-50%, about 35-45%, or about 35-40%) of the total lipids in a suitable lipid solution by weight or by molar.

In some embodiments, LNPs may further include a lipid conjugate. As used herein, the term “lipid conjugate” is meant to refer to a conjugated lipid that inhibits aggregation of LNPs. Such lipid conjugates include, but are not limited to, polyethylene glycol (PEG)-lipid conjugates such as, e.g., PEG coupled to dialkyloxypropyls (e.g, PEG-DAA conjugates), PEG coupled to diacylglycerols (e.g, PEG-DAG conjugates), PEG coupled to cholesterol, PEG coupled to phosphatidylethanolamines, and PEG conjugated to ceramides, ionizable PEG lipids, polyoxazoline (POZ)-lipid conjugates, polyamide oligomers (e.g, ATTA-lipid conjugates), and mixtures thereof. PEG or POZ can be conjugated directly to the lipid or may be linked to the lipid via a linker moiety. Any linker moiety suitable for coupling the PEG or the POZ to a lipid can be used including, e.g, non-ester containing linker moieties and ester-containing linker moieties. In certain preferred embodiments, non-ester containing linker moieties, such as amides or carbamates, are used.

In some embodiments, lipid conjugates include a PEG-modified lipid. In embodiments, the PEGylated lipid can be used to enhance lipid nanoparticle colloidal stability in vitro and circulation time in vivo. Illustrative PEG-lipids for use in LNPs include, but are not limited to PEG-c-DOMG, PEG-DMG, PEG-DLPE, PEG-DMPE, PEG-DPPE, PEG-DSG, PEG-DSPE, DiMystyrlGlycerol (DMG), 1,2-Dipalmitoyl-rac-glycerol, methoxypolyethylene Glycol (DPG-PEG), 1,2-Distearoyl-rac-glycero-3-methylpolyoxyethylene (DSG-PEG). In some embodiments, a lipid conjugate has an average molecular mass from about 500 Da to about 5000 Da.

Lipid conjugates may constitute at least about 1%, 2%, 3%, 4%, 5%, 6%, 7%, 8%, 9%, 10%, 15% or 20% of the total lipids in a suitable lipid solution by weight or by molar. In some embodiments, lipid conjugates constitute about 1-5% (e.g., about 1-2%, about 1-3%, about 1-4%, about 2-5%, about 2-4%, about 2-3%, about 3-5%, about 3-4%, or about 4-5%) of the total lipids in a suitable lipid solution by weight or by molar.

A suitable lipid solution may contain a mixture of desired lipids at various concentrations. For example, a suitable lipid solution may contain a mixture of desired lipids at a total concentration of or greater than about 0.1 mg/ml, 0.5 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10 mg/ml, 15 mg/ml, 20 mg/ml, 30 mg/ml, 40 mg/ml, 50 mg/ml, or 100 mg/ml. In some embodiments, a suitable lipid solution may contain a mixture of desired lipids at a total concentration ranging from about 0.1-100 mg/ml, 0.25-50 mg/ml, 1.0-20 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml. In some embodiments, a suitable lipid solution may contain a mixture of desired lipids at a total concentration up to about 100 mg/ml, 90 mg/ml, 80 mg/ml, 70 mg/ml, 60 mg/ml, 50 mg/ml, 40 mg/ml, 30 mg/ml, 20 mg/ml, 10 mg/ml, 5 mg/mL, 4 mg/mL, 3 mg/mL, 2 mg/mL, 1 mg/mL, 0.5 mg/mL, 0.25 mg/mL, or 0.1 mg/mL.

In some embodiments, the ionizable lipid is included in the lipid solution in a molar percentage from about 30%-60%, including 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and 60%, or any range having endpoints defined by any two of the aforementioned values. In embodiments, the helper lipid is included in the lipid solution in a molar percentage from about 0%-30%, including 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, and 30%, or any range having endpoints defined by any two of the aforementioned values. In embodiments, the sterol is included in the lipid solution in a molar percentage from about 25%-50%, including 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, and 50%, or any range having endpoints defined by any two of the aforementioned values. In embodiments, the lipid conjugate is included in the lipid solution in a molar percentage from about 0%-10%, including 1%, 2%, 3%, 4%, 5%, 6%, 7%, 8%, 9%, and 10%, or any range having endpoints defined by any two of the aforementioned values.

In some embodiments, the cargo includes a nucleic acid (e.g., DNA, RNA, e.g., mRNA). The nucleic acid may be at a concentration between 50 μg per ml and 200 μg per ml of the aqueous solution (e.g., about 50 μg/ml, about 60 μg/ml, about 70 μg/ml, about 80 μg/ml, about 90 μg/ml, about 100 μg/ml, about 110 μg/ml, about 120 μg/ml, about 130 μg/ml, about 140 μg/ml, about 150 μg/ml, about 175 μg/ml, or about 200 μg/ml). All of or a portion of the nucleic acid may be encapsulated in the lipid nanoparticles. In some embodiments, the method yields a nucleic acid encapsulation efficiency of at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or greater). In some embodiments, the method yields a nucleic acid encapsulation efficiency of at least 90%. In some embodiments, the method yields a nucleic acid encapsulation efficiency between about 90% and about 97%.

Generally, the LNPs are prepared at a molar ratio between the amine group of the ionizable lipid and the phosphate group of the mRNA, from about 5:1 to 60:1. In some embodiments, the lipid to nucleic acid ratio (mass/mass ratio; w/w ratio) can be in the range of from about 1:1 to about 60:1, from about 1:1 to about 20:1, from about 1:1 to about 19:1, from about 1:1 to about 18:1, from about 1:1 to about 17:1, from about 1:1 to about 16:1, from about 1:1 to about 15:1, from about 1:1 to about 14:1, from about 1:1 to about 13:1, from about 1:1 to about 12:1, from about 1:1 to about 10:1, from about 1:1 to about 9:1, from about 1:1 to about 8:1, from about 1:1 to about 7:1, from about 1:1 to about 6:1, from about 1:1 to about 5:1, from about 3:1 to about 15:1, from about 4:1 to about 15:1, from about 5:1 to about 15:1, about 6:1 to about 15:1, from about 7:1 to about 15:1, from about 8:1 to about 15:1, from about 9:1 to about 15:1, from about 5:1 to about 10:1, from about 6:1 to about 10:1, from about 7:1 to about 10:1, from about 8:1 to about 10:1, or from about 9:1 to about 10:1.

The LNPs can be prepared with the cargo at a volume ratio with the lipid solution, such that the lipid solution:cargo ratio is from about 1:1 to 10:1, including 1:1, 2:1, 3:1, 4:1, 5:1, 6:1, 7:1, 8:1, 9:1 and 10:1, or any range having endpoints defined by any two of the aforementioned values.

### EXAMPLES

The following examples are given by way of illustration and are in no way intended to limit the scope of the present disclosure.

### Example 1: Synthesis of Ionizable Imidazole Lipid

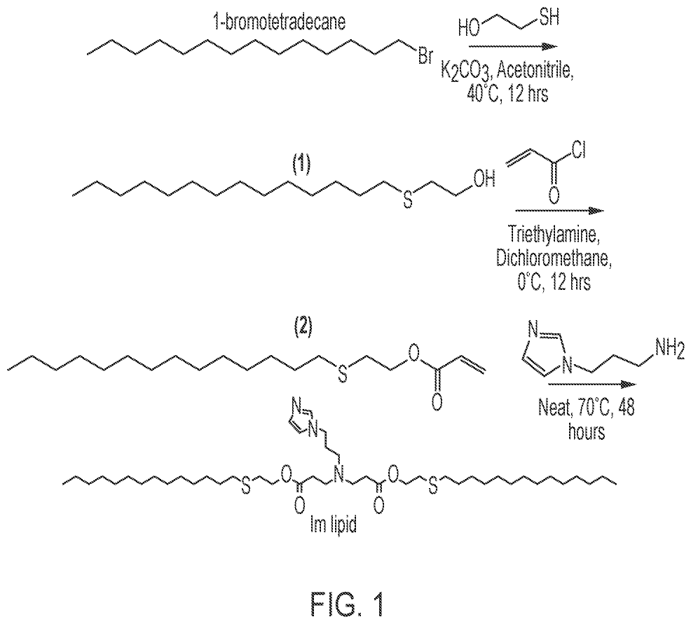

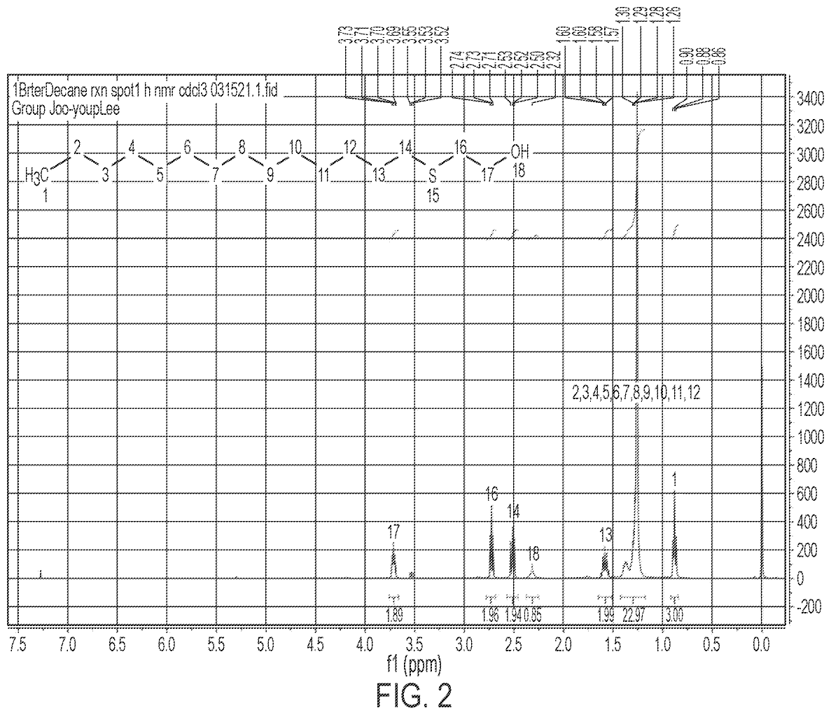

As depicted in FIG. 1, 1-bromotetradecane and potassium carbonate were mixed with a solution of 2-mercaptoethanol in acetonitrile. The mixture was stirred at 40° C. for twelve hours. Ethyl acetate was added to the mixture to extract Product 1. Product 1 was characterized by proton nuclear magnetic resonance on a 400 MHz spectrometer in CDCl₃, the results of which are depicted in FIG. 2. In particular, the data reported includes: chemical shift (δ ppm), multiplicity, integration, and coupling constant (Hz). ¹H NMR (400 MHz, CDCl₃) δ 3.71 (t, J=6.0 Hz, 2H), 2.73 (t, J=6.0 Hz, 2H), 2.60-2.45 (m, 2H), 2.32 (s, 1H), 1.58 (t, J=7.6 Hz, 2H), 1.26 (s, 22H), 0.88 (t, J=6.7 Hz, 3H).

Without further purification, Product 1 was added to a solution of trimethylamine in dichloromethane, with stirring. A solution of acryloyl chloride was added dropwise to the solution. The resulting mixture was stirred at 0° C. for twelve hours. Dichloromethane was added to extract Product 2.

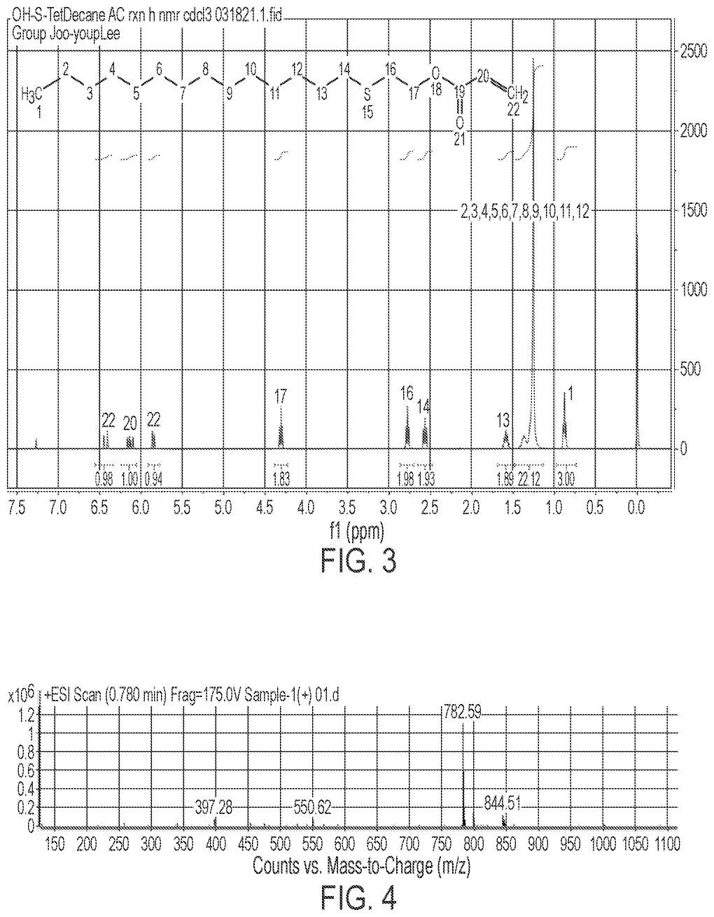

Product 2 was characterized by proton nuclear magnetic resonance on a 400 MHz spectrometer in CDCl₃, the results of which are depicted in FIG. 3. In particular, the data reported includes: chemical shift (δ ppm), multiplicity, integration, and coupling constant (Hz). ¹H NMR (400 MHz, CDCl₃) δ 6.43 (dd, J=17.4, 1.4 Hz, 1H), 6.13 (dd, J=17.3, 10.5 Hz, 1H), 5.85 (dd, J=10.4, 1.4 Hz, 1H), 4.31 (s, 2H), 2.78 (t, J=7.0 Hz, 2H), 2.57 (t, J=7.5 Hz, 2H), 1.59 (t, J=7.5 Hz, 2H), 1.26 (s, 22H), 0.88 (t, J=6.7 Hz, 3H).

Without further purification, Product 2 was added to 1-(3-aminopropyl) imidazole and allowed to react for 48 hours at 70° C., generating the final Im Lipid. The resultant Im Lipid was characterized by electrospray ionization mass spectrometry (ESI-MS), the results of which are depicted in FIG. 4. The m/z ratio calculated for the Im lipid [M+H]+ was 782.59; the observed was 782.59.

### Example 2: Preparation of Lipid Nanoparticles

Six lipid nanoparticles were formulated with ionizable lipids, DOPE, cholesterol and DMG-PEG. The ionizable lipids contained varying molar percentages of Im lipid and DLin-MC3-DMA (MC3), according to Table 2 below.

TABLE 2

| Percent Im | Percent MC3 |
|---|---|
| 100 | 0 |
| 90 | 10 |
| 75 | 25 |
| 50 | 50 |
| 25 | 75 |
| 0 | 100 |

The ionizable lipids, DOPE, cholesterol and DMG-PEG were dissolved in ethanol at a concentration of 2 mg/ml and were mixed at a molar ratio of 40:10:48:2 to create a lipid mixture.

A molar ratio of 8:1 between the amine group of the ionizable lipid and the phosphate group of mRNA (Trilink Biotechnologies, L-7701) was used to determine the amount of mRNA to be added. mRNA was diluted in 5 mM citrate buffer (pH 5.0) and was mixed with the lipid mixture at a 3:1 volume ratio.

The solution was incubated for 30 minutes at room temperature and the solution was concentrated using an Amicon filter (MWCO: 30,000 Da) to remove the ethanol.

The encapsulation efficiency was determined using the QuantiFluor® RNA system (Promega).

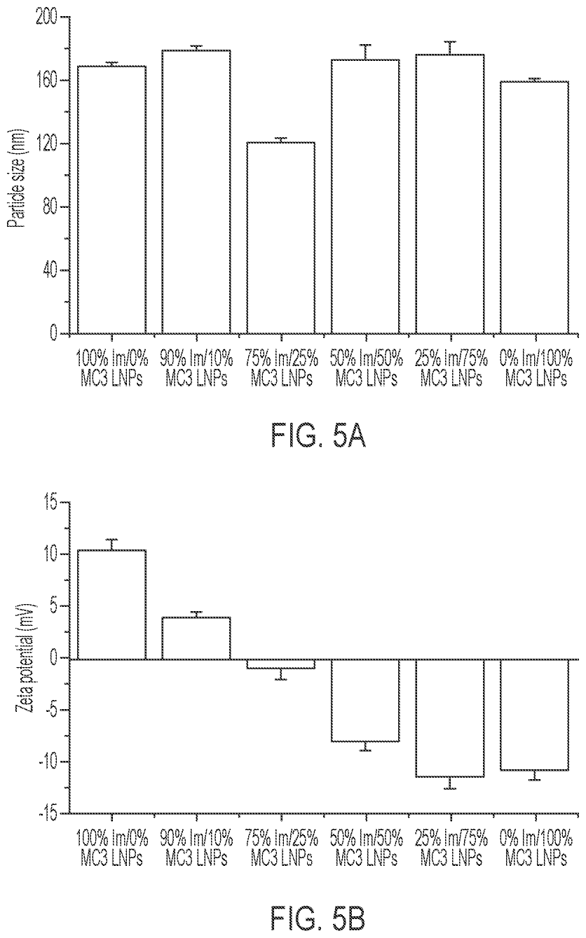

The LNPs were characterized for particle size and surface charge using dynamic light scattering and zeta potential measurements. The particle size, depicted in FIG. 5A, and the surface charge, depicted in FIG. 5B, were determined using Nanobrook Omni (Brookhaven Instruments, Holtsville, NY). The LNPs showed a range of particle sizes of ˜120-175 nm. The surface charge of the LNPs ranged from +10 mV to −10 mV. Since these LNPs contained ionizable lipids, their surface charge was close to neutral when they were measured at physiological pH of 7.4.

Flow cytometry was used to determine in vitro cellular uptake and transfection efficacy in both human Jurkat T cells and HeLa cells. The cells were seeded in a 96-well plate at a density of 40,000 cells/well. Different formulations containing 100 ng of mRNA were added to each well and were incubated for 18-20 hours at 37° C. The cells were washed (for suspension cells) or trypsinized (for adherent cells) and centrifuged at 300×g. After the cells were diluted in PBS, flow cytometry analysis was performed using the FL-1 and FL-4 channels to quantify the amounts of cellular uptake and transfection efficiency, respectively.

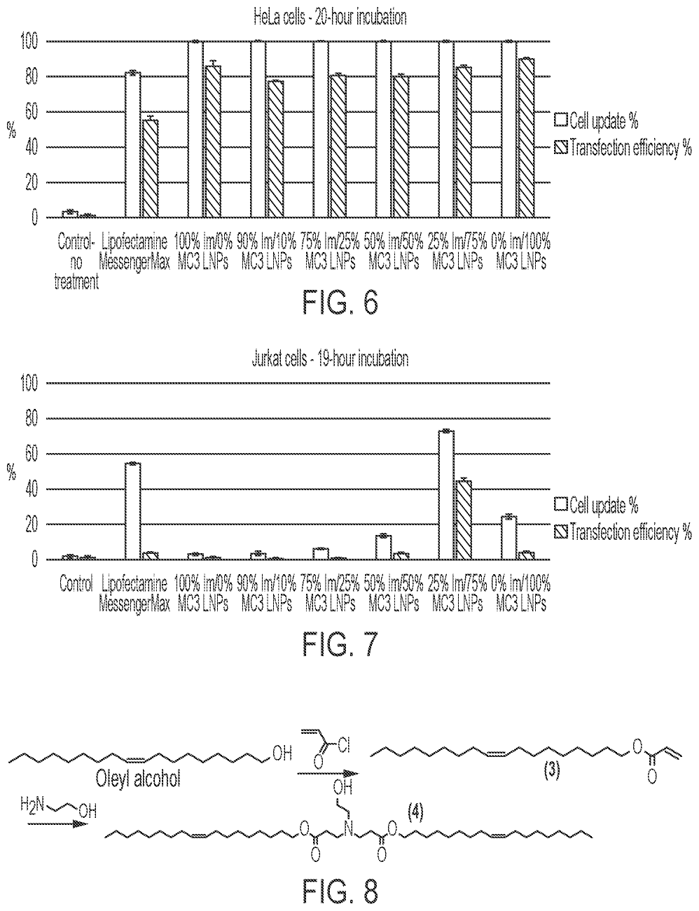

LNPs with different compositions were tested against HeLa and Jurkat cells for both cellular uptake and mRNA transfection efficiencies (FIGS. 6 and 7). HeLa cells are relatively easy to transfect with transfecting agents and thus were chosen as a representative cell line. Human Jurkat cells are representative T lymphocytes, which are known to be difficult to transfect. The LNPs were tested for cellular uptake using Cy5-labeled eGFP mRNA. Cy5 fluorescent dye was used to determine cellular uptake while eGFP fluorescence was used to determine the transfection efficiency of the mRNA encapsulated in the different LNP formulations. After 20-hour incubation, fluorescence was measured by flow cytometry. In all the different LNP formulations, ˜100% cellular uptake was obtained in HeLa cells. The positive control with Lipofectamine MessengerMax showed a cellular uptake efficacy of ˜82%. (FIG. 6) In Jurkat cells, the cellular uptake was ˜55% for the positive control, Lipofectamine MessengerMax. LNPs showed mixed results for the cellular uptake efficiencies in terms of different ionizable lipid ratios. Even the positive control with ˜55% cellular uptake showed ˜4% transfection efficiency.

Notably, as shown in FIG. 7, the 25% Im/75% MC3 LNP outperformed the positive control in both cellular uptake efficiency (˜70%) and transfection efficiency (˜45%) by a wide margin in the Jurkat cells.

### Example 3: Lipid 2AEOAP2

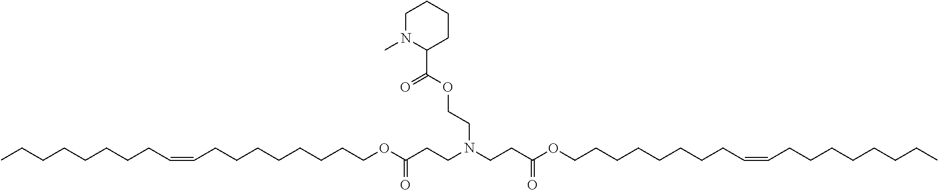 [SMILES: CCCCCCCC/C=C\CCCCCCCCOC(=O)CCN(CCOC(=O)C1CCCCN1C)CCC(=O)OCCCCCCCC/C=C\CCCCCCCC]

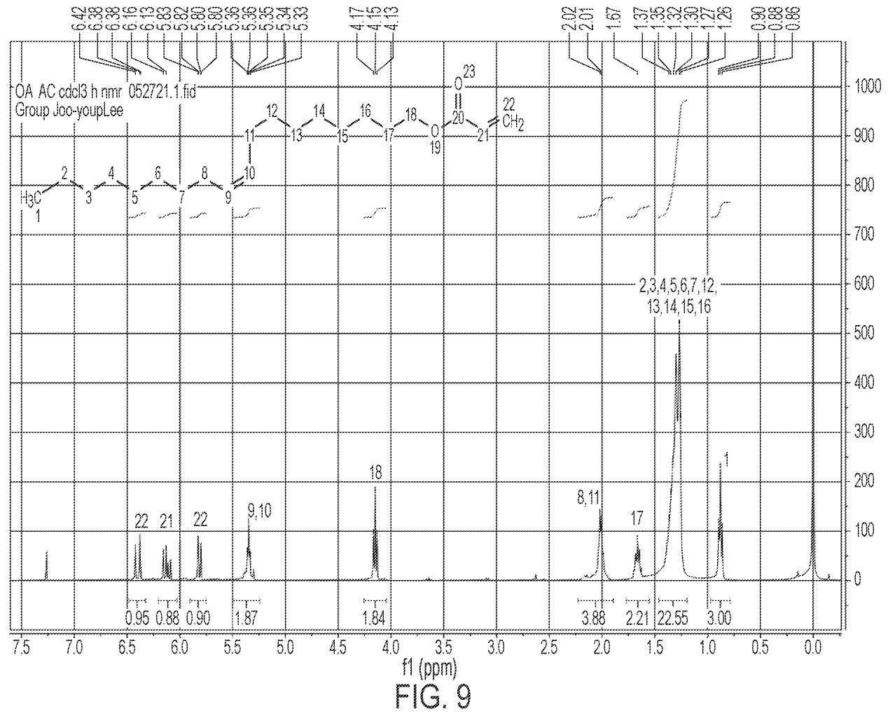

As depicted in FIG. 8, acryloyl chloride (1.5 equivalents) was added dropwise to a 2:1 solution of triethylamine in dichloromethane and oleyl alcohol, with stirring. The reaction was stirred overnight. DCM was added to the mixture to extract Product 3, (9Z)-9-Octadecen-1-yl 2-propenoate. Product 3 was dried to allow the solvent to evaporate. Product 3 was characterized by proton nuclear magnetic resonance on a 400 MHz spectrometer in CDCl₃, the results of which are depicted in FIG. 9. In particular, the data reported includes: chemical shift (δ ppm), multiplicity, integration, and coupling constant (Hz). ¹H NMR (400 MHz, CDCl₃) δ 6.48-6.33 (m, 1H), 6.14 (d, J=10.4 Hz, 1H), 5.81 (dd, J=10.4, 1.5 Hz, 1H), 5.50-5.24 (m, 2H), 4.15 (t, J=6.7 Hz, 2H), 2.01 (d, J=6.2 Hz, 3H), 1.67 (s, 2H), 1.30 (dt, J=17.4, 10.5 Hz, 22H), 0.88 (t, J=6.6 Hz, 3H).

Product 3 (1 eq.) was stirred with 2-aminoethanol (0.6 eq.) at 70° C. for 48 hours. The reaction was monitored using thin layer chromatography (TLC). Water and DCM were added to extract the reaction product. The solvent was evaporated to yield Product 4, β-Alanine, N-(2-hydroxyethyl)-N-[3-(9-octadecenyloxy)-3-oxopropyl]-, 9-octadecenyl ester, (Z,Z)-(9CI).

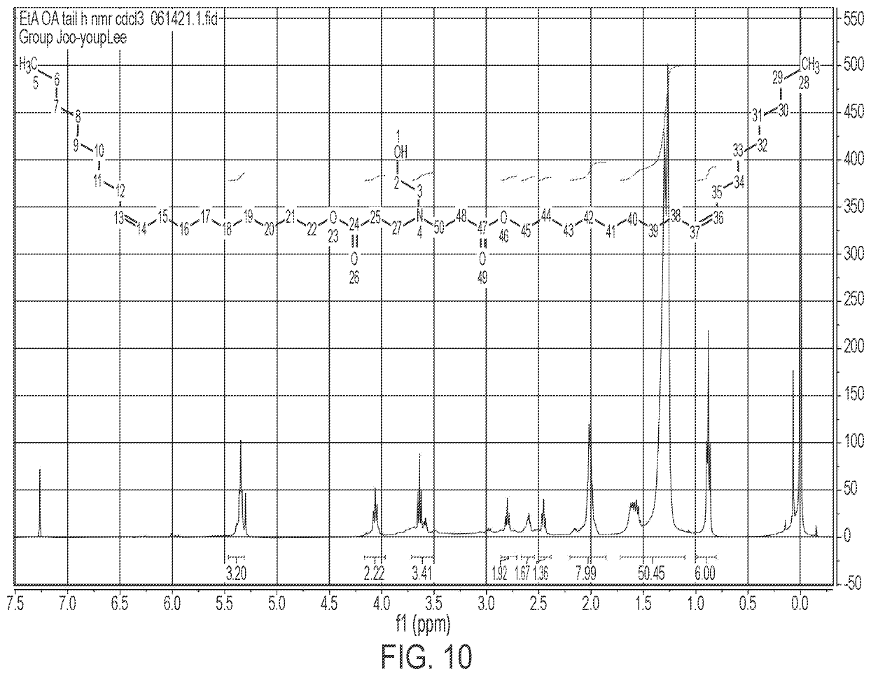

Product 4 was characterized by proton nuclear magnetic resonance on a 400 MHz spectrometer in CDCl₃, the results of which are depicted in FIG. 10. In particular, the data reported includes: chemical shift (δ ppm), multiplicity, integration, and coupling constant (Hz). ¹H NMR (400 MHz, CDCl₃) δ 5.46-5.23 (m, 4H), 4.05 (q, J=8.3, 7.6 Hz, 2H), 3.76-3.51 (m, 4H), 2.80 (t, J=6.8 Hz, 4H), 2.67-2.54 (m, 4H), 2.45 (t, J=6.8 Hz, 2H), 2.01 (q, J=6.5 Hz, 8H), 1.65-1.17 (m, 49H), 0.88 (t, J=6.6 Hz, 6H).

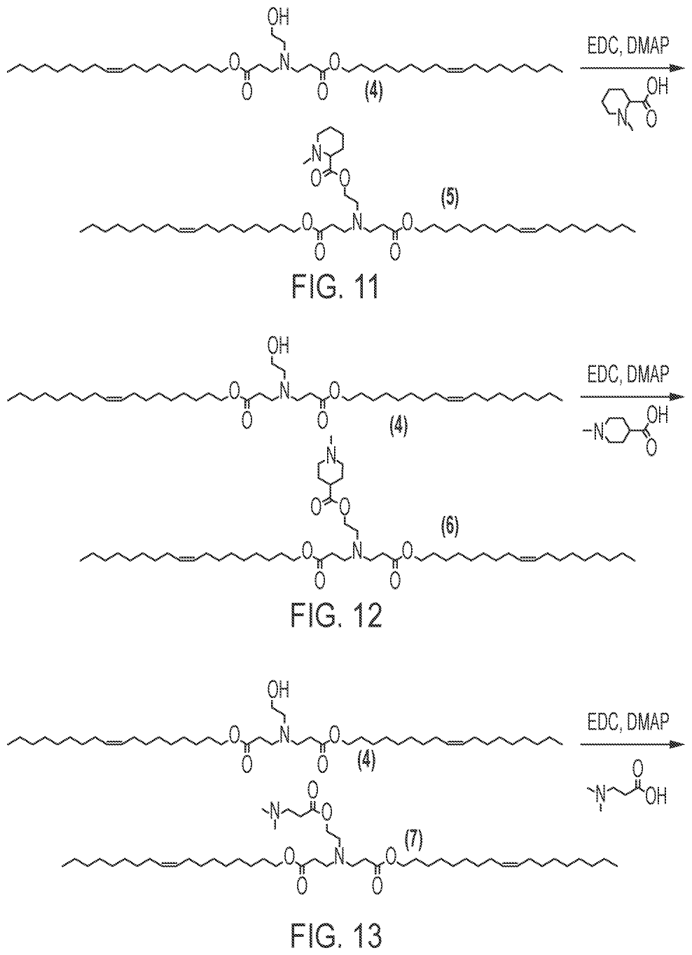

As depicted in FIG. 11, Product 4 (1 eq.) was dissolved in a solution of DCM and dimethylformamide (DMF). 1-Ethyl-3-(3-dimethylaminopropyl) carbodiimide (EDC) (1.8 eq.) and 4-Dimethylaminopyridine (DMAP) (0.4 eq.) were added to the solution, with stirring. 1-Methylpiperidine-2-carboxylic acid hydrochloride (1.5 eq.) was added. After stirring overnight, the solvent was reduced using a rotary evaporator to yield Product 5, ionizable lipid 2AEOAP2.

### Example 4: Lipid 2AEOAP4

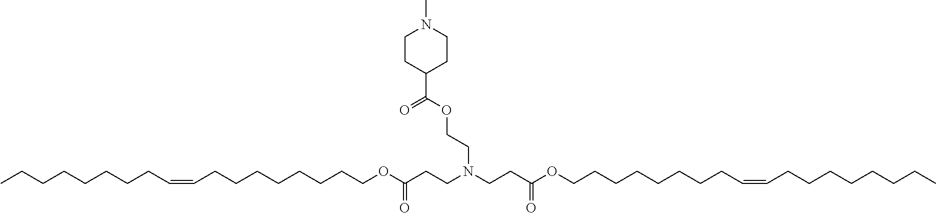 [SMILES: CCCCCCCC/C=C\CCCCCCCCOC(=O)CCN(CCOC(=O)C1CCN(C)CC1)CCC(=O)OCCCCCCCC/C=C\CCCCCCCC]

As depicted in FIG. 12, Product 4 (1 eq.) was dissolved in a solution of DCM and DMF. EDC (1.8 eq.) and DMAP (0.4 eq.) were added to the solution, with stirring. 1-Methyl-4-piperidinecarboxylic acid (1.5 eq.) was added. After stirring overnight, the solvent was reduced using a rotary evaporator to yield Product 6, ionizable lipid 2AEOAP4.

### Example 5: Lipid 2AEOAD2

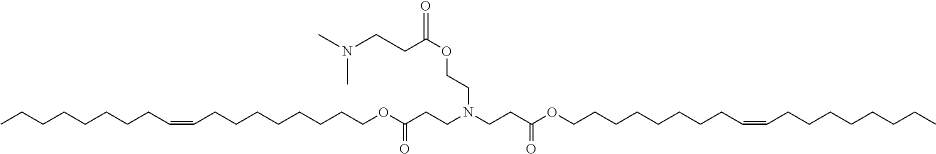 [SMILES: CCCCCCCC/C=C\CCCCCCCCOC(=O)CCN(CCOC(=O)CCN(C)C)CCC(=O)OCCCCCCCC/C=C\CCCCCCCC]

As depicted in FIG. 13, Product 4 (1 eq.) was dissolved in a solution of DCM and DMF. EDC (1.8 eq.) and DMAP (0.4 eq.) were added to the solution, with stirring. 3-(Dimethylamino) propionic acid hydrochloride (1.5 eq.) was added. After stirring overnight, the solvent was reduced using a rotary evaporator to yield Product 7, ionizable lipid 2AEOAD2.

### Example 6: Lipid 2AEOAD3

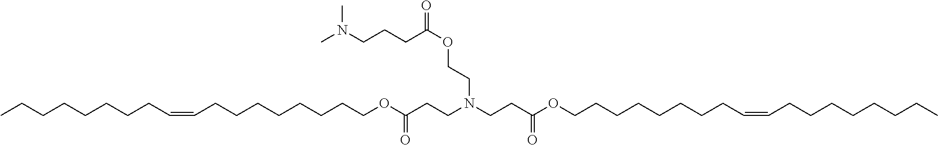 [SMILES: CCCCCCCC/C=C\CCCCCCCCOC(=O)CCN(CCOC(=O)CCCN(C)C)CCC(=O)OCCCCCCCC/C=C\CCCCCCCC]

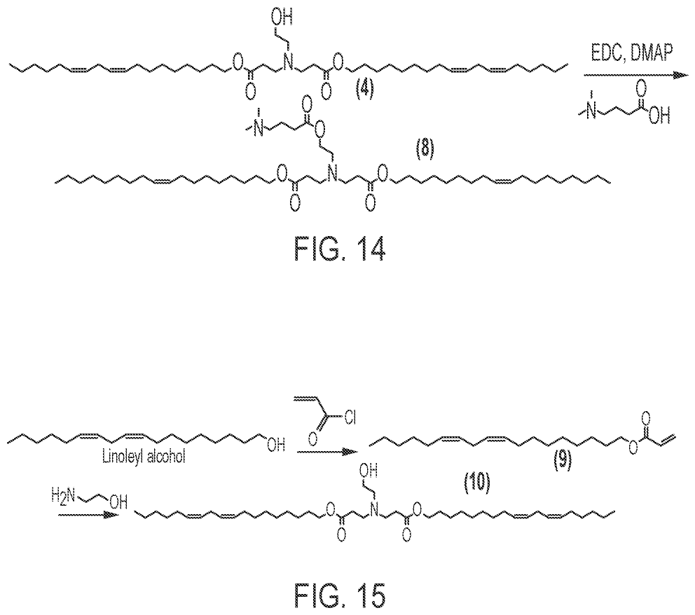

As depicted in FIG. 14, Product 4 (1 eq.) was dissolved in a solution of DCM and DMF. EDC (1.8 eq.) and DMAP (0.4 eq.) were added to the solution, with stirring. 4-dimethylaminobutyric acid hydrochloride (1.5 eq.) was added. After stirring overnight, the solvent was reduced using a rotary evaporator to yield Product 8, ionizable lipid 2AEOAD3.

### Example 7: Lipid 2AELAP2

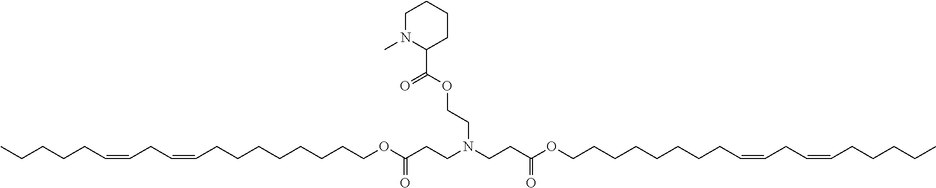 [SMILES: CCCCC/C=C\C/C=C\CCCCCCCCOC(=O)CCN(CCOC(=O)C1CCCCN1C)CCC(=O)OCCCCCCCC/C=C\C/C=C\CCCCC]

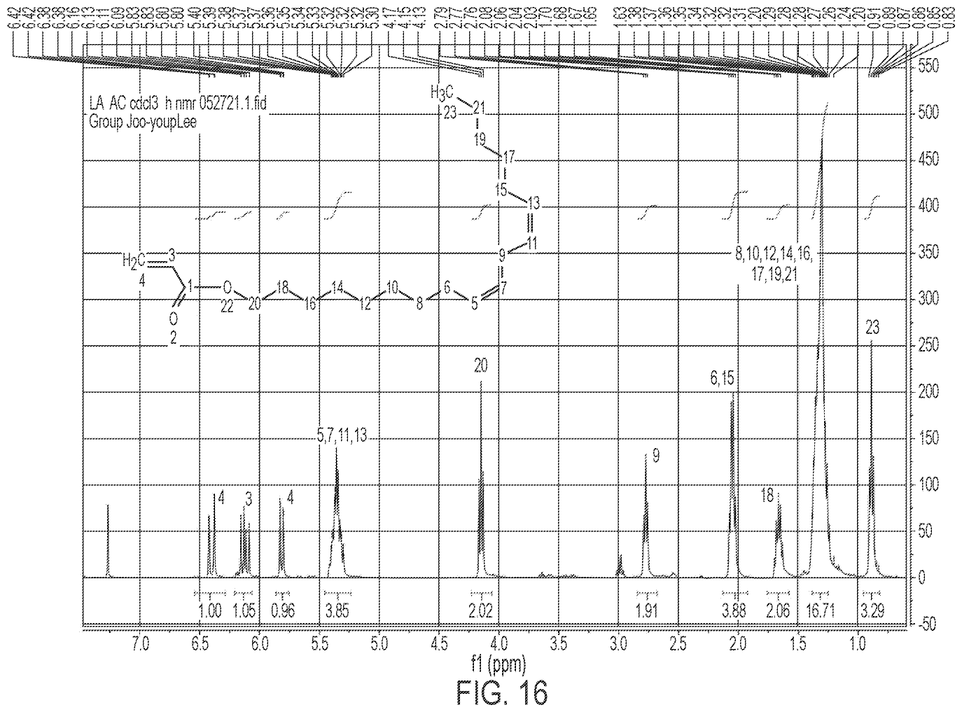

As depicted in FIG. 15, acryloyl chloride (1.5 equivalents) was added dropwise to a 2:1 solution of trrethylamine in dichloromethane and linoleyl alcohol, with stirring. The reaction was stirred overnight. DCM was added to the mixture to extract Product 9, 2-propenoic acid, 9,12-octadecadienyl ester, (Z,Z)-(9CI). Product 9 was dried to allow the solvent to evaporate. Product 9 was characterized by proton nuclear magnetic resonance on a 400 MHz spectrometer in CDCl₃, the results of which are depicted in FIG. 16. In particular, the data reported includes: chemical shift (δ ppm), multiplicity, integration, and coupling constant (Hz). ¹H NMR (400 MHz, CDCl₃) δ 6.40 (dd, J=17.3, 1.5 Hz, 1H), 6.12 (dd, J=17.3, 10.4 Hz, 1H), 5.81 (dd, J=10.4, 1.5 Hz, 1H), 5.46-5.25 (m, 4H), 4.15 (t, J=6.7 Hz, 2H), 2.77 (t, J=6.5 Hz, 2H), 2.05 (q, J=6.8 Hz, 4H), 1.67 (p, J=6.8 Hz, 2H), 1.31 (qt, J=13.7, 5.4 Hz, 16H), 0.89 (t, J=6.7 Hz, 3H).

Product 9 (1 eq.) was stirred with 2-aminoethanol (0.6 eq.) at 70° C. for 48 hours. The reaction was monitored using thin layer chromatography (TLC). Water and DCM were added to extract the reaction product. The solvent was evaporated to yield Product 10:

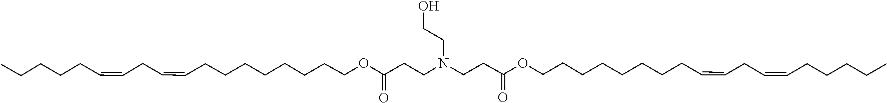 [SMILES: CCCCC/C=C\C/C=C\CCCCCCCCOC(=O)CCN(CCO)CCC(=O)OCCCCCCCC/C=C\C/C=C\CCCCC]

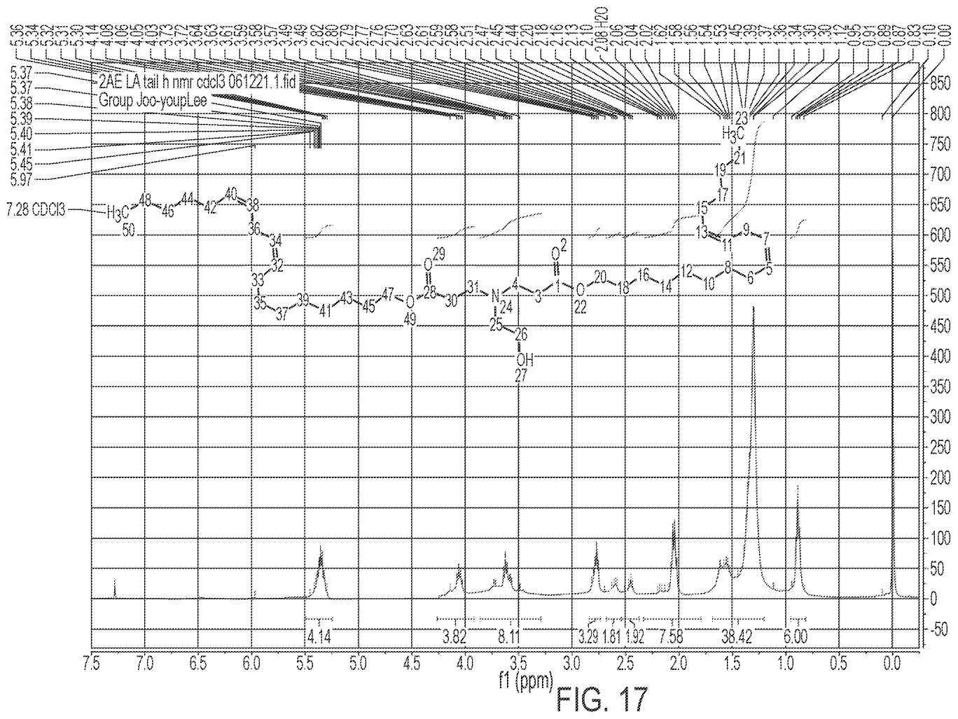

Product 10 was characterized by proton nuclear magnetic resonance on a 400 MHz spectrometer in CDCl₃, the results of which are depicted in FIG. 17. In particular, the data reported includes: chemical shift (δ ppm), multiplicity, integration, and coupling constant (Hz). ¹H NMR (400 MHz, CDCl₃) δ 5.35 (tt, J=11.1, 4.7 Hz, 8H), 4.05 (d, J=6.8 Hz, 2H), 3.64 (t, J=6.7 Hz, 4H), 2.96-2.31 (m, 14H), 2.05 (q, J=6.9 Hz, 8H), 1.65-1.25 (m, 37H), 0.88 (d, J=7.0 Hz, 6H).

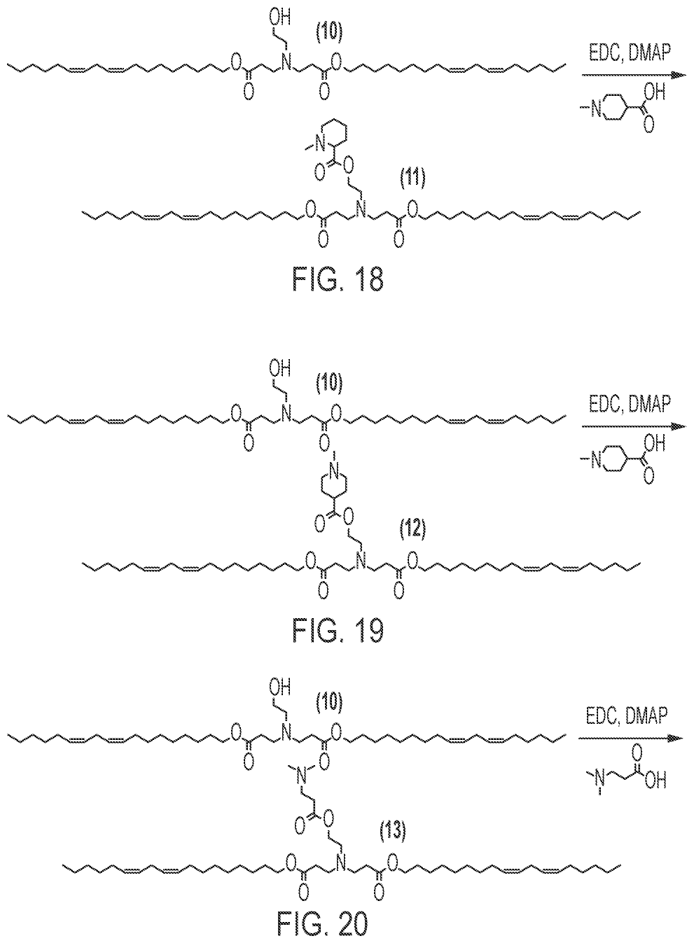

As depicted in FIG. 18, Product 10 (1 eq.) was dissolved in a solution of DCM and DMF. EDC (1.8 eq.) and DMAP (0.4 eq.) were added to the solution, with stirring. 1-methylpiperidine-2-carboxylic acid hydrochloride (1.5 eq.) was added. After stirring overnight, the solvent was reduced using a rotary evaporator to yield Product 11, ionizable lipid 2AELAP2.

### Example 8: Lipid 2AELAP4

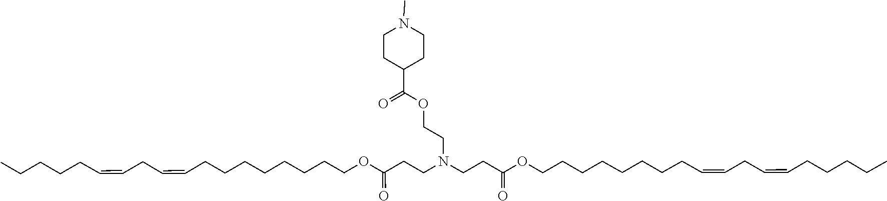 [SMILES: CCCCC/C=C\C/C=C\CCCCCCCCOC(=O)CCN(CCOC(=O)C1CCN(C)CC1)CCC(=O)OCCCCCCCC/C=C\C/C=C\CCCCC]

As depicted in FIG. 19, Product 10 (1 eq.) was dissolved in a solution of DCM and DMF. EDC (1.8 eq.) and DMAP (0.4 eq.) were added to the solution, with stirring. 1-methyl-4-piperidinecarboxylic acid (1.5 eq.) was added. After stirring overnight, the solvent was reduced using a rotary evaporator to yield Product 12, ionizable lipid 2AELAP4.

### Example 9: Lipid 2AELAD2

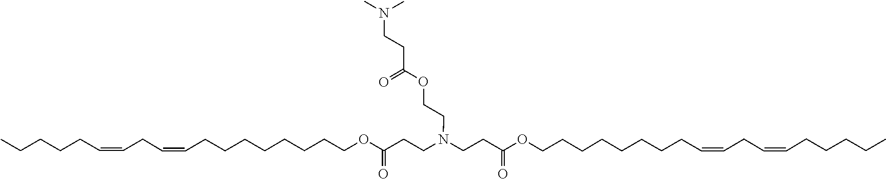 [SMILES: CCCCC/C=C\C/C=C\CCCCCCCCOC(=O)CCN(CCOC(=O)CCN(C)C)CCC(=O)OCCCCCCCC/C=C\C/C=C\CCCCC]

As depicted in FIG. 20, Product 10 (1 eq.) was dissolved in a solution of DCM and DMF. EDC (1.8 eq.) and DMAP (0.4 eq.) were added to the solution, with stirring. 3-(dimethylamino) propionic acid hydrochloride (1.5 eq.) was added. After stirring overnight, the solvent was reduced using a rotary evaporator to yield Product 13, ionizable lipid 2AELAD2.

### Example 10: Lipid 2AELAD3

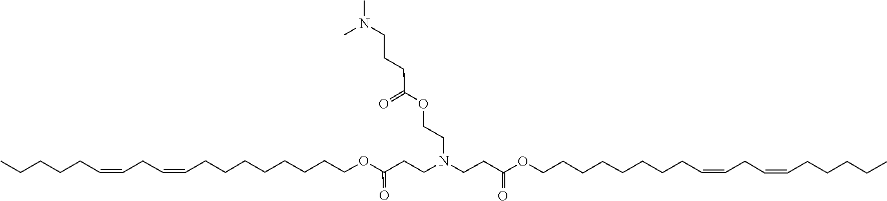 [SMILES: CCCCC/C=C\C/C=C\CCCCCCCCOC(=O)CCN(CCOC(=O)CCCN(C)C)CCC(=O)OCCCCCCCC/C=C\C/C=C\CCCCC]

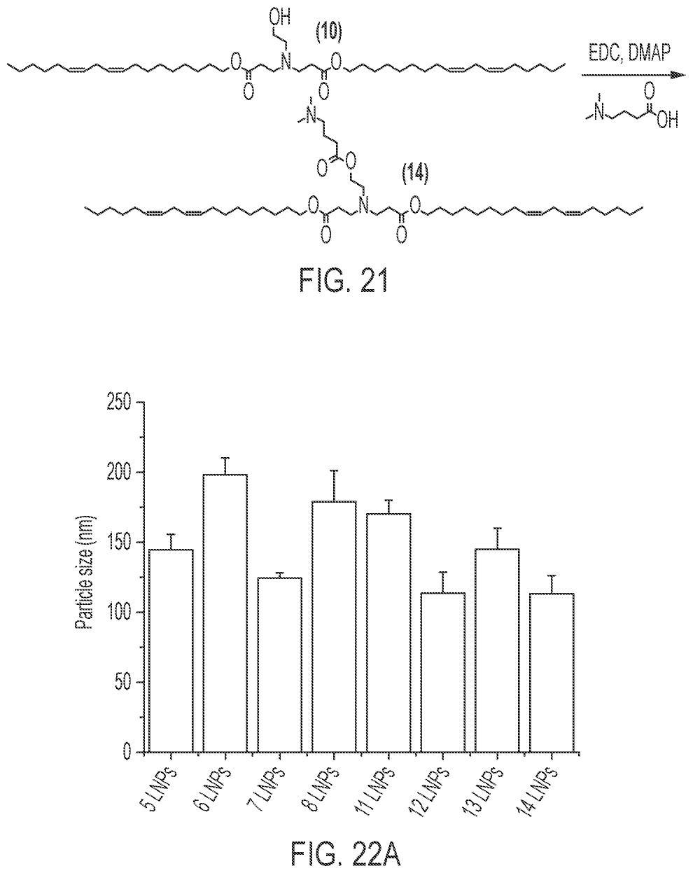

As depicted in FIG. 21, Product 10 (1 eq.) was dissolved in a solution of DCM and DMF. EDC (1.8 eq.) and DMAP (0.4 eq.) were added to the solution, with stirring. 4-dimethylaminobutyric acid hydrochloride (1.5 eq.) was added. After stirring overnight, the solvent was reduced using a rotary evaporator to yield Product 14, ionizable lipid 2AELAD3.

### Example 11: Lipids 16A-16D

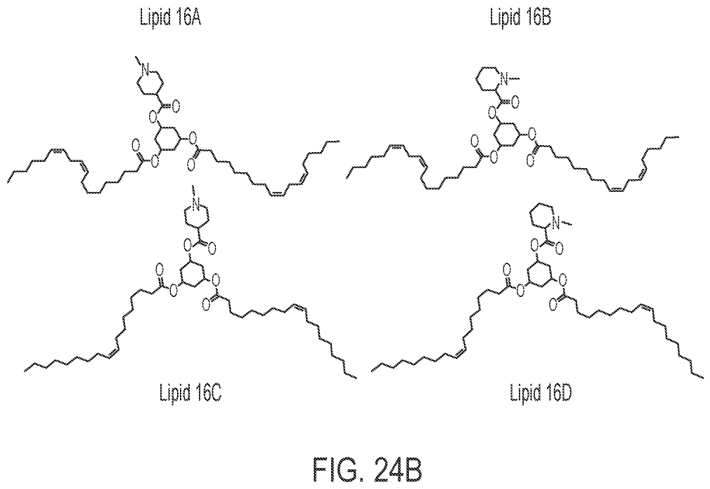

As depicted in FIG. 24A, N,N′-dicyclohexyl carbodiimide and 4-dimethylaminopyridine was added to a solution of cyclohexane-1,3,5-triol in dichloromethane. A solution of 1-methylpiperidine-2-carboxylic acid hydrochloride or 1-methyl-4-piperidinecarboxylic acid was added and stirred overnight. The reaction mixture was monitored using thin layer chromatography to make sure the reactant was completely consumed. The reaction was extracted using ethyl acetate and the desired Product 15 was separated using column chromatography. After confirming the structure of Product 15 using NMR and MS, Product 15 was used to synthesize Products 16A-D.

To synthesize Product 16, Product 15 was stirred with N,N′-dicyclohexyl carbodiimide and 4-dimethylaminopyridine. Oleic acid or linoleic acid was added and the reaction was stirred overnight. The reaction mixture was extracted and the desired product was separated using column chromatography. The resultant products (Products 16A-D) are depicted in FIG. 24B.

### Example 12: Lipids 20A-20B

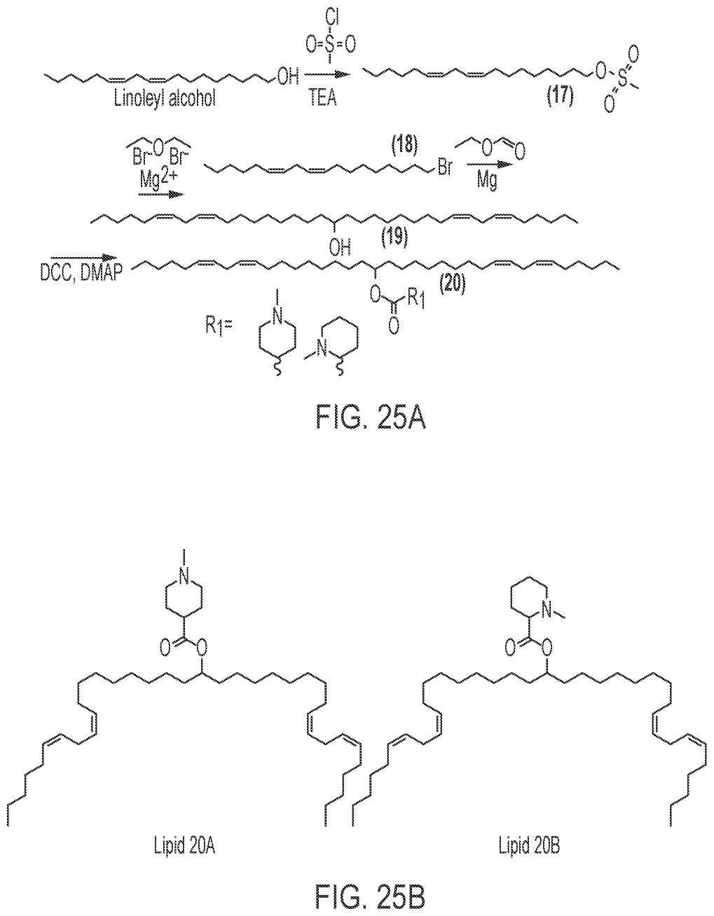

As shown in FIG. 25A, to synthesize Product 17, linoleyl alcohol was reacted with methanesulfonyl chloride in the presence of triethylamine (TEA) in dichloromethane (DCM). The reaction mixture was extracted, and Product 17 was confirmed using NMR and MS. Magnesium bromide ethyl etherate was added to a solution of Product 17 in diethyl ether. The reaction mixture was extracted, generating Product 18, which was confirmed using NMR and MS.

Magnesium turnings were added to a dry flask, followed by diethyl ether. Product 18 was added dropwise, generating an exothermic reaction. Iodine may be added to initiate the reaction. After the reaction is completed, ethyl formate was added dropwise. The reaction mixture was extracted, and Product 19 was separated by column chromatography.

The reaction system was degassed using nitrogen and reflux condensation. Product 19 was stirred with N,N′-dicyclohexyl carbodiimide, and 4-dimethylaminopyridine. 1-Methylpiperidine-2-carboxylic acid hydrochloride was added to produce Lipid 20A.

Product 19 was stirred with N, N′-dicyclohexyl carbodiimide, and 4-dimethylaminopyridine. 1-methyl-4-piperidinecarboxylic acid was added to produce Lipid 20B.

Product 19 was stirred with N, N′-dicyclohexyl carbodiimide, and 4-dimethylaminopyridine. 1H-Imidazol-1-ylacetic acid was added to produce Lipid 20C.

### Example 13: Preparation of Lipid Nanoparticles Using an Ethanol Dilution Method

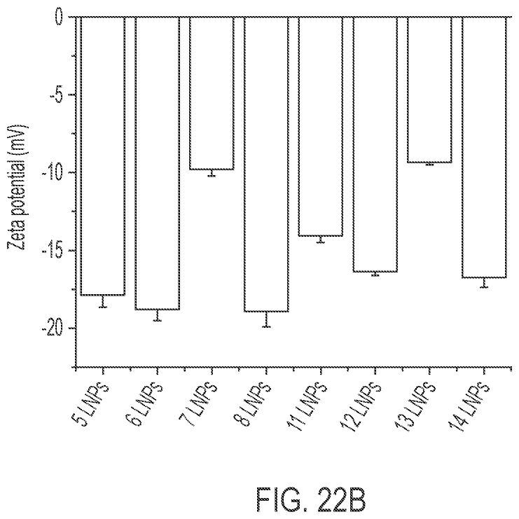

Different lipid nanoparticles (LNPs) were formulated by incorporating the different ionizable lipids from Table 1. DLin-MC3-DMA (MC3) LNPs were prepared as a control. The lipids were formulated with the ionizable lipids, DOPE, cholesterol, and DMG-PEG. Each of the lipids were dissolved in ethanol at a concentration of 2 mg/ml. The composition of ionizable lipid varied from 40-60%, DOPE ranged from 10-20%, cholesterol ranged from 30-50% and DMG-PEG ranged from 1-5%. A molar ratio of ranging from 5:1 to 15:1 was used between the amine group of the ionizable lipid and the phosphate group of the mRNA. mRNA diluted in 5 mM citrate buffer (pH 5.0) was mixed with the lipid mixture at a 3:1 volume ratio. After incubating the sample for 30 minutes, the solution was concentrated using an Amicon® filter (MilliporeSigma, Burlington, MA, MWCO: 30,000 Da) to remove the ethanol. The encapsulation efficiency of mRNA was determined using QuantiFluor® RNA system (Promega). Particle size and surface charge of the LNPs were determined using Nanobrook Omni, the results of which are demonstrated in FIGS. 22A-22B.

The LNPs were characterized for particle size and surface charge using dynamic light scattering and zeta potential measurements (FIGS. 22A-22B). LNPs showed a range of particle size of from ˜100 to ˜200 nm (FIG. 22A). The surface charge of the LNPs ranged from −10 mV to −20 mV (FIG. 22B). Since these LNPs contained ionizable lipids, their surface charge was close to neutral when they were measured at physiological pH of 7.4.

### Example 14: In Vitro Cellular Uptake and Transfection Efficiencies

Flow cytometry was used to determine in vitro cellular uptake and transfection efficacy in human Jurkat T cells. The cells were seeded in a 96-well plate at a density of 40,000 cells/well. Different formulations containing 100 ng of mRNA were added to each well and were incubated for 21 hours at 37° C. The cells were washed and centrifuged at 300×g. After the cells were diluted in PBS, flow cytometry analysis was performed to quantify the amounts of cellular uptake and transfection efficiency.

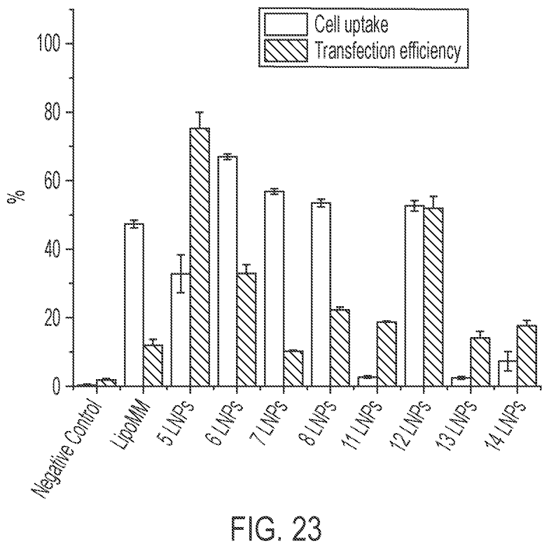

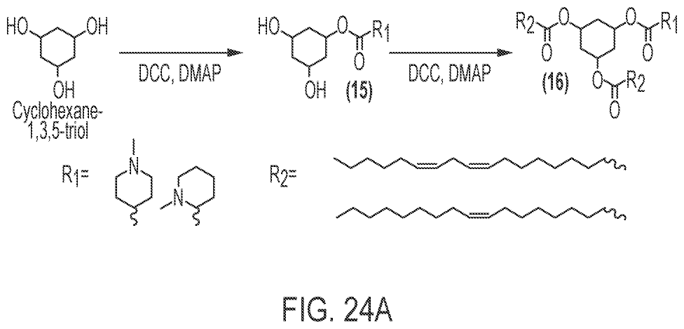

LNPs with different compositions were tested against Jurkat cells for both cellular uptake and mRNA transfection efficiencies, as depicted in FIG. 23. Human Jurkat cells are representative T lymphocytes which are known to be difficult to transfect. The LNPs were tested for cellular uptake using Cy5-labeled eGFP mRNA. Cy5 fluorescent dye was used to determine the cellular uptake while eGFP fluorescence was used to determine the transfection efficiency of the mRNA encapsulated in the different LNP formulations. After 21-hour incubation, fluorescence was measured using a flow cytometer. The cellular uptake varied from ˜2% to ˜90%. MC3 LNPs, used as a positive control, showed a cell uptake of 90%and a transfection efficiency ˜92%. LNPs showed mixed results for the cellular uptake efficiencies in terms of different ionizable lipid ratios. The transfection efficiencies for the different LNPs varied ˜10% to a maximum of ˜75%. The LNPs with the maximum transfection efficiencies were identified to be 2AEOAP2 LNPs, 2AEOAP4 LNPs, 2AELAP4 LNPs.

### Further Examples

A first item of the present disclosure, either alone or in combination with any other item herein, concerns a composition comprising at least one ionizable lipid according to Formula (I) or a pharmaceutically-acceptable salt thereof, in which a) R1 is independently selected from

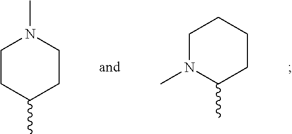

and R2 is selected from

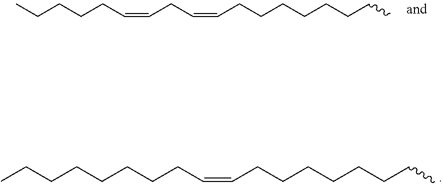

A second item of the present disclosure, either alone or in combination with any other item herein, concerns the composition of the first item, wherein the ionizable lipid is selected from the group consisting of: 2AEOAP2, 2AEOAP4, 2AELAP2, and 2AELAP4.

A third item of the present disclosure, either alone or in combination with any other item herein, concerns a composition comprising at least one ionizable lipid according to Formula (II), or a pharmaceutically-acceptable salt thereof, in which a) R1 is independently selected from

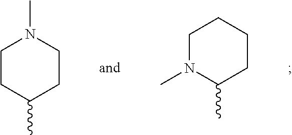

and b) R2 is selected from

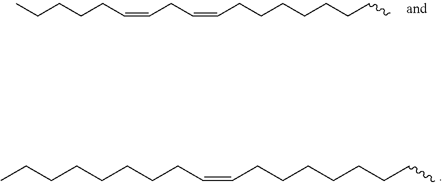

A fourth item of the present disclosure, either alone or in combination with any other item herein, concerns the composition of the third item, wherein the ionizable lipid is selected from the group consisting of Lipid 16A, Lipid 16B, Lipid 16C, and Lipid 16D.

A fifth item of the present disclosure, either alone or in combination with any other item herein, concerns a composition comprising Lipid 20B.

A sixth item of the present disclosure, either alone or in combination with any other item herein, concerns the composition of any of the previous compositions, further comprising: a helper lipid; a sterol; and a PEGylated lipid conjugate, wherein the composition forms lipid nanoparticles.

A seventh item of the present disclosure, either alone or in combination with any other item herein, concerns the composition of the sixth item, wherein the ionizable lipid is selected from the group consisting of: 2AEOAP2, 2AEOAP4, 2AELAP2, 2AELAP4, Lipid 16A, Lipid 16B, Lipid 16C, Lipid 16B, and Lipid 20B.

An eighth item of the present disclosure, either alone or in combination with any other item herein, concerns the composition of the sixth item, wherein the helper lipid is selected from the group consisting of: 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dioleyl-sn-glycero-3-phosphotidylcholine (DOPC) 1,2-dipalmitoyl-sn-glycero-3-phosphoethanolamine (DPPE), 1,2-dimyristoyl-sn-glycero-3-phosphoethanolamine (DMPE), and 1,2-dioleoyl-sn-glycero-3-phospho-(1′-rac-glycerol) (DOPG).

A ninth item of the present disclosure, either alone or in combination with any other item herein, concerns the composition of the sixth item, wherein the helper lipid is DOPE.

A tenth item of the present disclosure, either alone or in combination with any other item herein, concerns the composition of the sixth item, wherein the sterol is cholesterol or a derivative thereof.

An eleventh item of the present disclosure, either alone or in combination with any other item herein, concerns the composition of the sixth item, wherein the PEGylated lipid conjugate is PEGylated myristoyl diglyceride (PEG-DMG).

A twelfth item of the present disclosure, either alone or in combination with any other item herein, concerns the composition of the sixth item, wherein the lipid nanoparticle at least partially encapsulates a nucleic acid.

A thirteenth item of the present disclosure, either alone or in combination with any other item herein, concerns the composition of the twelfth item, wherein the nucleic acid is mRNA.

A fourteenth item of the present disclosure, either alone or in combination with any other item herein, concerns the composition of any of the previous items, further comprising a pharmaceutically acceptable excipient.

A fifteenth item of the present disclosure, either alone or in combination with any other item herein, concerns the composition of any of the previous items, wherein the composition is formulated for administration by injection or infusion.

A sixteenth item of the present disclosure, either alone or in combination with any other item herein, concerns the composition of the sixth item, wherein the ionizable lipid comprises from about 40-60% mole percent, the helper lipid comprises from about 10-20% molar percent, the sterol comprises from about 30-50% mole percent; and the conjugate lipid comprises from about 1-5% mole percent.

A seventeenth item of the present disclosure, either alone or in combination with any other item herein, concerns the use of the composition of any of the previous items in a subject.

An eighteenth item of the present disclosure, either alone or in combination with any other item herein, concerns the use of the seventeenth item, wherein the subject is a mammal.

A nineteenth item of the present disclosure, either alone or in combination with any other item herein, concerns the use of the seventeenth item, wherein the subject is a human.

A twentieth item of the present disclosure, either alone or in combination with any other item herein, concerns the use of the seventeenth item, as a vaccine component.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A composition comprising at least one ionizable lipid according to Formula (I), 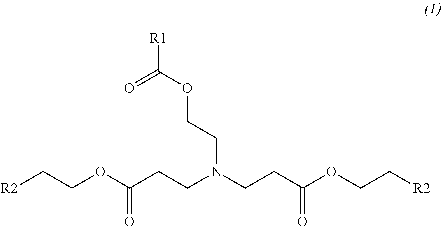
or a pharmaceutically-acceptable salt thereof, in which:
R1 is independently selected from 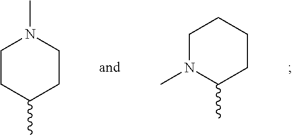
and R2 is selected from

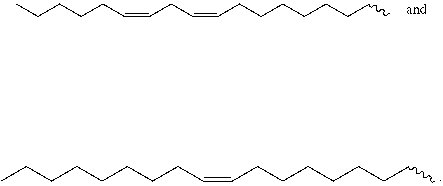

2. The composition of claim 1, wherein the ionizable lipid is selected from the group consisting of:

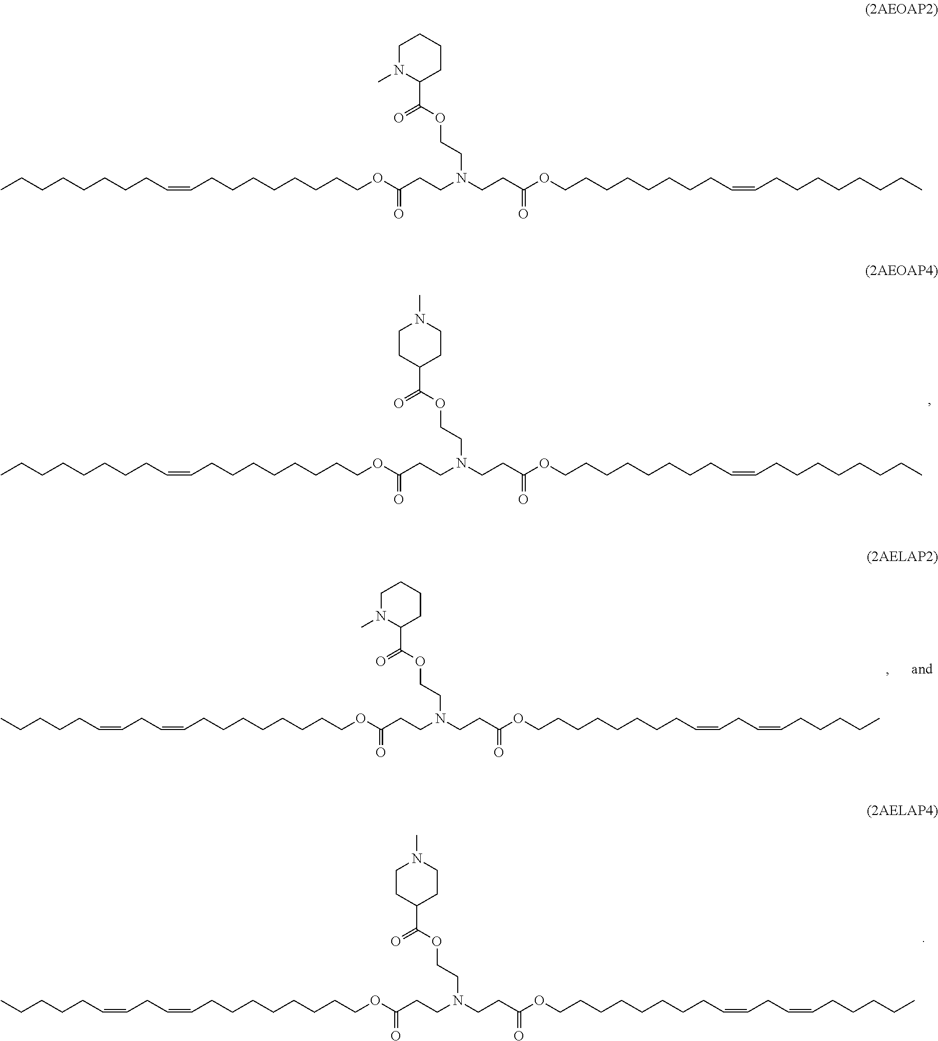

3. A composition comprising at least one ionizable lipid according to Formula (II), 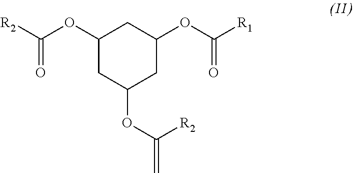
or a pharmaceutically-acceptable salt thereof, in which:
R1 is independently selected from and 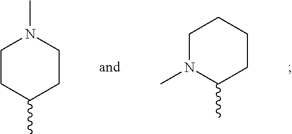
and R2 is selected from

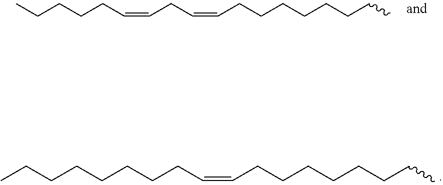

4. The composition of claim 3, wherein the ionizable lipid is selected from the group consisting of:

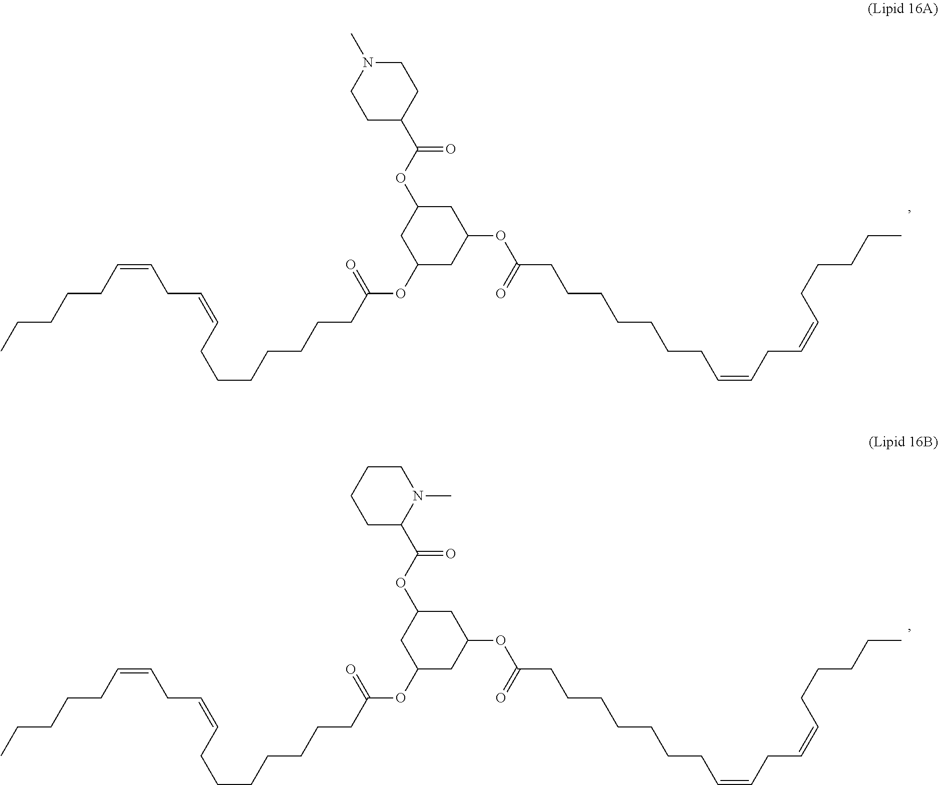

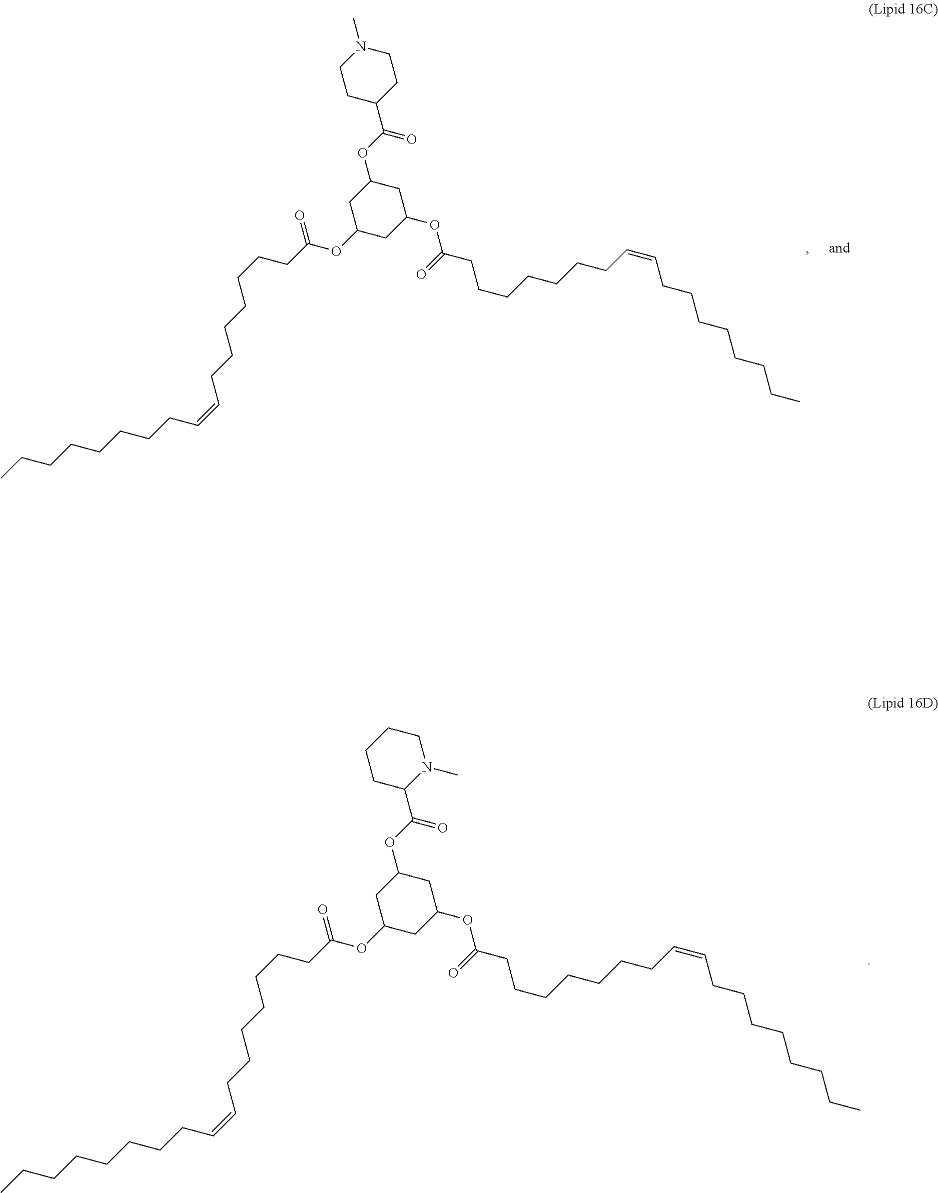

5. A composition comprising at least one ionizable lipid having the structure:

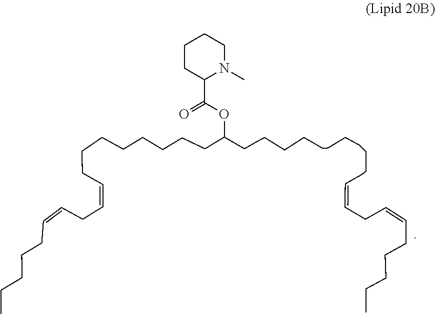 [SMILES: CCCCC/C=C\C/C=C\CCCCCCCCCCCCC(CCCCCCCC/C=C\C/C=C\CCCCC)OC(=O)C1CCCCN1C]

6. The composition of claim 1, further comprising:
a helper lipid;
a sterol; and
a PEGylated lipid conjugate,
wherein the composition forms lipid nanoparticles.

7. The composition of claim 6, wherein the ionizable lipid is selected from the group consisting of: 2AEOAP2, 2AEOAP4, 2AELAP2, and 2AELAP4.

8. The composition of claim 6, wherein the helper lipid is selected from the group consisting of: 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dioleyl-sn-glycero-3-phosphotidylcholine (DOPC) 1,2-dipalmitoyl-sn-glycero-3-phosphoethanolamine (DPPE), 1,2-dimyristoyl-sn-glycero-3-phosphoethanolamine (DMPE), and 1,2-dioleoyl-sn-glycero-3-phospho-(1′-rac-glycerol) (DOPG).

9. The composition of claim 6, wherein the helper lipid is DOPE.

10. The composition of claim 6, wherein the sterol is cholesterol or a derivative thereof.

11. The composition of claim 6, wherein the PEGylated lipid conjugate is PEGylated myristoyl diglyceride (PEG-DMG).

12. The composition of claim 6, wherein the lipid nanoparticle at least partially encapsulates a nucleic acid.

13. The composition of claim 12, wherein the nucleic acid is mRNA.

14. The composition of claim 6, further comprising a pharmaceutically acceptable excipient.

15. The composition of claim 14, wherein the composition is formulated for administration by injection or infusion.

16. The composition of claim 6, wherein the ionizable lipid comprises from about 40-60% mole percent, the helper lipid comprises from about 10-20% molar percent, the sterol comprises from about 30-50% mole percent; and the conjugate lipid comprises from about 1-5% mole percent.

17. The composition of claim 3, further comprising:
a helper lipid;
a sterol; and
a PEGylated lipid conjugate,
wherein the composition forms lipid nanoparticles.

18. The composition of claim 5, further comprising:
a helper lipid;
a sterol; and
a PEGylated lipid conjugate,
wherein the composition forms lipid nanoparticles.
